SEQUENCE LISTING (31 sequences)

SEQ ID NO: 1 - protein, 112 aa, Mus sp.
GPELVKPGASVKISCKASGYAFSYSWMNWVKLRPGQGLEWIGRIFPGDGDTDYNGKFKGKATLTADKSSNTAYMQLTSLTSVDSAVYLCARNVFDGYWLVYWGQGTLVTVSA

SEQ ID NO: 2 - protein, 103 aa, Mus sp.
NPVTLGTSASISCRSSKSLLHSNGITYLYWYLQKPGQSPQLLIYQMSNLVSGVPDRFSSSGSGTDFTLRISRVEAEDVGVYYCAQNLELPYTFGGGTKLEIKR

SEQ ID NO: 3 - protein, 119 aa, Artificial Sequence
  Description of Artificial Sequence Synthetic polypeptide
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 4 - protein, 119 aa, Artificial Sequence
  Description of Artificial Sequence Synthetic polypeptide
QVQLVQSGAEVKKPGSSVKVSCKASGYAFSYSWMNWVRQAPGQGLEWMGRIFPGDGDTDYNGKFKGRVTITADKSTSTAYMELSSLRSEDTAVYLCARNVFDGYWLVYWGQGTLVTVSS

SEQ ID NO: 5 - protein, 119 aa, Artificial Sequence
  Description of Artificial Sequence Synthetic polypeptide
QVQLVQSGAEVKKPGASVKVSCKVSGYAFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 6 - protein, 119 aa, Artificial Sequence
  Description of Artificial Sequence Synthetic polypeptide
QVQLVQSGAEVKKPGSSVKVSCKASGYAFSYSWMSWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 7 - protein, 119 aa, Artificial Sequence
  Description of Artificial Sequence Synthetic polypeptide
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 8 - protein, 119 aa, Artificial Sequence
  Description of Artificial Sequence Synthetic polypeptide
QVQLVQSGAEVKKPGSSVKVSCKASGYAFSYSWISWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 9 - protein, 119 aa, Artificial Sequence
  Description of Artificial Sequence Synthetic polypeptide
QVQLVQSGAEVKKPGASVKVSCKASGYTFTYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 10 - protein, 119 aa, Artificial Sequence
  Description of Artificial Sequence Synthetic polypeptide
QVQLVQSGAEVKKPGASVKVSCKASGYTFSYSWMNWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 11 - protein, 119 aa, Artificial Sequence
  Description of Artificial Sequence Synthetic polypeptide
EVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 12 - protein, 119 aa, Artificial Sequence
  Description of Artificial Sequence Synthetic polypeptide
EVQLVESGGGLVKPGGSLRLSCAASGFAFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 13 - protein, 119 aa, Artificial Sequence
  Description of Artificial Sequence Synthetic polypeptide
QVQLVESGGGLVKPGGSLRLSCAASGFTFSYSWMNWVRQAPGKGLEWVGRIFPGDGDTDYNGKFKGRVTITADKSTSTAYMELSSLRSEDTAVYYCARNVFDGYWLVYWGQGTLVTVSS

SEQ ID NO: 14 - protein, 119 aa, Artificial Sequence
  Description of Artificial Sequence Synthetic polypeptide
EVQLVESGAGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 15 - protein, 119 aa, Artificial Sequence
  Description of Artificial Sequence Synthetic polypeptide
EVQLVESGGGVVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 16 - protein, 119 aa, Artificial Sequence
  Description of Artificial Sequence Synthetic polypeptide
EVQLVESGGGLKKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 17 - protein, 119 aa, Artificial Sequence
  Description of Artificial Sequence Synthetic polypeptide
EVQLVESGGGLVKPGSSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 18 - protein, 119 aa, Artificial Sequence
  Description of Artificial Sequence Synthetic polypeptide
EVQLVESGGGLVKPGGSLRVSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 19 - protein, 119 aa, Artificial Sequence
  Description of Artificial Sequence Synthetic polypeptide
EVQLVESGGGLVKPGGSLRLSCAASGFTFSYSWMNWVRQAPGKGLEWMGRIFPGDGDTDYNGKFKGRVTITADKSTSTAYMELSSLRSEDTAVYYCARNVFDGYWLVYWGQGTLVTVSS

SEQ ID NO: 20 - protein, 115 aa, Artificial Sequence
  Description of Artificial Sequence Synthetic polypeptide
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV

SEQ ID NO: 21 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 22 - protein, 144 aa, Homo sapiens [sequence omitted]

SEQ ID NO: 23 - protein, 152 aa, Homo sapiens
MSRLPVLLLLQLLVRPGLQAPMTQTTPLKTSWVNCSNMIDEIITHLKQPPLPLLDFNNLNGEDQDILMENNLRRPNLEAFNRAVKSLQNASAIESILKNLLPCLPLATAAPTRHPIHIKDGDWNEFRRKLTFYLKTLENAQAQQTTLSLAIF

SEQ ID NO: 24 - protein, 152 aa, Homo sapiens [sequence omitted]

SEQ ID NO: 25 - protein, 152 aa, Homo sapiens [sequence omitted]

SEQ ID NO: 26 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSSSPQLQESVFHLLVPSVILVLLAVGGLLFYRWRRRSHQEPQRADSPLEQPEGSPLTQDDRQVELPV

SEQ ID NO: 27 - protein, 554 aa, Homo sapiens
MTAPGAAGRCPPTTWLGSLLLLVCLLASRSITEEVSEYCSHMIGSGHLQSLQRLIDSQMETSCQITFEFVDQEQLKDPVCYLKKAFLLVQDIMEDTMRFRDNTPNAIAIVQLQELSLRLKSCFTKDYEEHDKACVRTFYETPLQLLEKVKNVFNETKNLLDKDWNIFSKNCNNSFAECSSQDVVTKPDCNCLYPKAIPSSDPASVSPHQPLAPSMAPVAGLTWEDSEGTEGSSLLPGEQPLHTVDPGSAKQRPPRSTCQSFEPPETPVVKDSTIGGSPQPRPSVGAFNPGMEDILDSAMGTNWVPEEASGEASEIPVPQGTELSPSRPGGGSMQTEPARPSNFLSASSPLPASAKGQQPADVTGTALPRVGPVRPTGQDWNHTPQKTDHPSALLRDPPEPGSPRISSLRPQGLSNPSTLSAQPQLSRSHSSGSVLPLGELEGRRSTRDRRSPAEPEGGPASEGAARPLPRFNSVPLTDTGHERQSEGSFSPQLQESVFHLLVPSVILVLLAVGGLLFYRWRRRSHQEPQRADSPLEQPEGSPLTQDDRQVELPV

SEQ ID NO: 28 - protein, 120 aa, Artificial Sequence [sequence omitted]

SEQ ID NO: 29 - protein, 108 aa, Artificial Sequence
  Description of Artificial Sequence Synthetic polypeptide
DIQMTQSPSSLSASVGDRVTITCRASQGINNYLNWYQQKPGKAPKRLIYNTNNLQTGVPSRFSGSGSGTEFTLTISSLQPEDFATYYCLQHNSFPTFGQGTKLEIKRT

SEQ ID NO: 30 - protein, 118 aa, Artificial Sequence
  Description of Artificial Sequence Synthetic polypeptide
QVELVESGGGVVQPGRSQRLSCAASGFTFSSYGMHWVRQAPGKGLEWVAIIWFDGSSTYYADSVRGRFTISRDNSKNTLYLQMNSLRAEDTAVYFCARELGRRYFDLWGRGTLVSVSS

SEQ ID NO: 31 - protein, 108 aa, Artificial Sequence
  Description of Artificial Sequence Synthetic polypeptide
EIVLTQSPATLSLSPGERATLSCRASQSVSSYLAWYQQKPGQAPRLLIYDASKRATGIPARFSGSGSGTDFTLTISSLEPEDFAVYYCQQRSKWPPWTFGQGTKVESK

(19) United States
(12) Patent Application Publication
(10) Pub. No.: US 2010/0247484 A1
(43) Pub. Date: Sep. 30, 2010

(54) COMBINATION THERAPY OF AN AFUCOSYLATED ANTIBODY AND ONE OR MORE OF THE CYTOKINES GM CSF, M CSF AND/OR IL3

(76) Inventors: HEINRICH BARCHET, BERNRIED (DE); GEORG FERTIG, PENZBERG (DE); CLAUDIA KIRSTENPFAD, PENZBERG (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/748,484

(22) Filed: Mar. 29, 2010

(30) Foreign Application Priority Data
Mar. 31, 2009 (EP) 09004754.9

(51) Int. Cl. A61K 38/20; A61K 39/395; A61K 38/19; A61P 35/04

(52) U.S. Cl. 424/85.2; 424/133.1

(57) ABSTRACT

The present invention is directed to the combination therapy of an afucosylated antibody specifically binding to a tumor-antigen with one or more cytokines selected from the group of human GM-CSF, human M-CSF and/or human IL-3 for the treatment of cancer.

### PRIORITY TO RELATED APPLICATION(S)

This application claims the benefit of European Patent Application No. 09004754.9, filed Mar. 31, 2009, which is hereby incorporated by reference in its entirety.

The instant application contains a Sequence Listing copy in ASCII format which has been submitted via EFS-Web and is hereby incorporated by reference in its entirety. Said ASCII copy, created on Feb. 23, 2010, is named 25390.txt, and is 43,980 bytes in size.

The present invention is directed to the combination treatment of a patient suffering from cancer with an afucosylated antibody specifically binding to a tumor-antigen and one or more of the cytokines human GM-CSF, human M-CSF and human IL-3, especially to the combination treatment of a patient suffering from monocytes/pericytes-infiltrated cancers.

### BACKGROUND OF THE INVENTION

### Afucosylated Antibodies

Cell-mediated effector functions of monoclonal antibodies can be enhanced by engineering their oligosaccharide component as described in Umaña, P., et al., Nature Biotechnol. 17 (1999) 176-180; and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A. and Morrison, S. L., Trends Biotechnol. 15 (1997) 26-32). Umaña, P., et al., Nature Biotechnol. 17 (1999) 176-180 and WO 99/54342 showed that overexpression in Chinese hamster ovary (CHO) cells of β(1,4)-N-acetylglucosaminyltransferase III (“GnTIII”), a glycosyltransferase catalyzing the formation of bisected oligosaccharides, significantly increases the in vitro ADCC activity of antibodies. Alterations in the composition of the N297 carbohydrate or its elimination affect also binding to Fc binding to FcγR and C1 q (Umaña, P., et al., Nature Biotechnol. 17 (1999) 176-180; Davies, J., et al., Biotechnol. Bioeng. 74 (2001) 288-294; Mimura, Y., et al., J. Biol. Chem. 276 (2001) 45539-45547; Radaev, S., et al., J. Biol. Chem. 276 (2001) 16478-16483; Shields, R. L., et al., J. Biol. Chem. 276 (2001) 6591-6604; Shields, R. L., et al., J. Biol. Chem. 277 (2002) 26733-26740; Simmons, L. C., et al., J. Immunol. Methods 263 (2002) 133-147).

Iida, S., et al., Clin. Cancer Res. 12 (2006) 2879-2887 show that efficacy of a afucosylated anti-CD20 antibody was inhibited by addition of fucosylated anti-CD20. The efficacy of a 1:9 mixture (10 microg/mL) of afucosylated and fucosylated anti-CD20s was inferior to that of a 1,000-fold dilution (0.01 microg/mL) of afucosylated anti-CD20 alone. They conclude that afucosylated IgG1, not including fucosylated counterparts, can evade the inhibitory effect of plasma IgG on ADCC through its high FcgammaRIIIa binding. Natsume, A., et al., shows in J. Immunol. Methods 306 (2005) 93-103 that fucose removal from complex-type oligosaccharide of human IgG1-type antibody results in a great enhancement of antibody-dependent cellular cytotoxicity (ADCC). Satoh, M., et al., Expert Opin. Biol. Ther. 6 (2006) 1161-1173 discusses afocusylated therapeutic antibodies as next-generation therapeutic antibodies. Satoh, M., concludes that antibodies consisting of only the afocusylated human IgG1 form is thought to be ideal. Kanda, Y., et al., Biotechnol. Bioeng. 94 (2006) 680-688 compared fucosylated CD20 antibody (96% fucosylation, CHO/DG44 1H5) with afocusylated CD20 antibody. Davies, J., et al., Biotechnol. Bioeng. 74 (2001) 288-294 reports that for a CD20 antibody increased ADCC correlates with increased binding to FcγRIII.

Methods to enhance cell-mediated effector functions of monoclonal antibodies by reducing the amount of fucose are described e.g. in WO 2005/018572, WO 2006/116260, WO 2006/114700, WO 2004/065540, WO 2005/011735, WO 2005/027966, WO 1997/028267, US 2006/0134709, US 2005/0054048, US 2005/0152894, WO 2003/035835, WO 2000/061739, Niwa, R., et al., J. Immunol. Methods 306 (2005) 151-160; Shinkawa, T. et al, J Biol Chem, 278 (2003) 3466-3473; WO 03/055993 or US 2005/0249722.

### CD20 and Anti CD20 Antibodies

The CD20 molecule (also called human B-lymphocyte-restricted differentiation antigen or Bp35) is a hydrophobic transmembrane protein with a molecular weight of approximately 35 kD located on pre-B and mature B lymphocytes (Valentine, M. A., et al. J. Biol. Chem. 264(19) (1989) 11282-11287; and Einfield, D. A., et al. (1988) EMBO J. 7(3):711-717; Tedder, T. F., et al., Proc. Natl. Acad. Sci. U.S.A. 85 (1988) 208-12; Stamenkovic, I., et al., J. Exp. Med. 167 (1988) 1975-80; Tedder, T. F., et al., J. Immunol. 142 (1989) 2560-8). CD20 is found on the surface of greater than 90% of B cells from peripheral blood or lymphoid organs and is expressed during early pre-B cell development and remains until plasma cell differentiation. CD20 is present on both normal B cells as well as malignant B cells. In particular, CD20 is expressed on greater than 90% of B cell non-Hodgkin's lymphomas (NHL) (Anderson, K. C., et al., Blood 63(6) (1984) 1424-1433)) but is not found on hematopoietic stem cells, pro-B cells, normal plasma cells, or other normal tissues (Tedder, T. F., et al., J, Immunol. 135(2) (1985) 973-979).

The 85 amino acid carboxyl-terminal region of the CD20 protein is located within the cytoplasm. The length of this region contrasts with that of other B cell-specific surface structures such as IgM, IgD, and IgG heavy chains or histocompatibility antigens class I1 a or β chains, which have relatively short intracytoplasmic regions of 3, 3, 28, 15, and 16 amino acids, respectively (Komaromy, M., et al., NAR 11 (1983) 6775-6785). Of the last 61 carboxyl-terminal amino acids, 21 are acidic residues, whereas only 2 are basic, indicating that this region has a strong net negative charge. The GenBank Accession No. is NP-690605. It is thought that CD20 might be involved in regulating an early step(s) in the activation and differentiation process of B cells (Tedder, T. F., et al., Eur. J. Immunol. 16 (8) (1986) 881-887) and could function as a calcium ion channel (Tedder, T. F., et al., J. Cell. Biochem. 14D (1990) 195).

There exist two different types of anti-CD20 antibodies differing significantly in their mode of CD20 binding and biological activities (Cragg, M. S., et al., Blood, 103 (2004) 2738-2743; and Cragg, M. S., et al., Blood, 101 (2003) 1045-1052). Type I antibodies, as e.g. rituximab (a non-afocusylated, non-glycoengineered antibody with normal glycosylation pattern), are potent in complement mediated cytotoxicity, whereas type II antibodies, as e.g. Tositumomab (B1), 11B8, AT80 or humanized B-Ly1 antibodies, effectively initiate target cell death via caspase-independent apoptosis with concomitant phosphatidylserine exposure.

The sharing common features of type I and type II anti-CD20 antibodies are summarized in Table 1.

TABLE 1
Properties of type I and type II anti-CD20 antibodies

| type I anti-CD20 antibodies | type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon | Strong cell death induction without |
| cross-linking | cross-linking |

U.S. Pat. No. 5,736,137 relates to Rituximab which is a non-afocusylated, non-glycoengineered antibody with normal glycosylation pattern. WO 2005/044859 and WO 2007/031875 relate to afocusylated anti-CD20 antibodies with a reduced amount of fucose compared to the corresponding parent antibodies. WO 2008/121876 (A2,A3) relate to afocusylated anti-CD20 antibodies with a reduced amount of fucose compared to the corresponding parent antibodies.

### EGFR and EFGR Antibodies

Human epidermal growth factor receptor (also known as HER-1 or Erb-B1, and referred to herein as “EGFR”) is a 170 kDa transmembrane receptor encoded by the c-erbB proto-oncogene, and exhibits intrinsic tyrosine kinase activity (Modjtahedi, H., et al., Br. J. Cancer 73 (1996) 228-235; Herbst, R. S., and Shin, D. M., Cancer 94 (2002) 1593-1611). SwissProt database entry P00533 provides the sequence of EGFR. There are also isoforms and variants of EGFR (e.g., alternative RNA transcripts, truncated versions, polymorphisms, etc.) including but not limited to those identified by Swissprot database entry numbers P00533-1, P00533-2, P00533-3, and P00533-4. EGFR is known to bind ligands including epidermal growth factor (EGF), transforming growth factor-α (TGf-α), amphiregulin, heparin-binding EGF (hb-EGF), betacellulin, and epiregulin (Herbst, R. S., and Shin, D. M., Cancer 94 (2002) 1593-1611; Mendelsohn, J., and Baselga, J., Oncogene 19 (2000) 6550-6565). EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay, G., et al., Ann. Oncology 14 (2003) 1346-1363; Tsao, A. S., and Herbst, R. S., Signal 4 (2003) 4-9; Herbst, R. S., and Shin, D. M., Cancer 94 (2002) 1593-1611; Modjtahedi, H., et al., Br. J. Cancer 73 (1996) 228-235).

Several murine monoclonal antibodies have been generated which achieve such a block in vitro and which have been evaluated for their ability to affect tumor growth in mouse xenograft models (Masui, H., et al., Cancer Res. 46 (1986) 5592-5598; Masui, H., et al., Cancer Res. 44 (1984) 1002-1007; Goldstein, N., et al., Clin. Cancer Res. 1 (1995) 1311-1318). For example, EMD 55900 (EMD Pharmaceuticals) is a murine anti-EGFR monoclonal antibody that was raised against human epidermoid carcinoma cell line A431 and was tested in clinical studies of patients with advanced squamous cell carcinoma of the larynx or hypopharynx (Bier, H., et al., Eur. Arch. Otohinolaryngol. 252 (1995) 433-9). In addition, the rat monoclonal antibodies ICR16, ICR62, and ICR80, which bind the extracellular domain of EGFR, have been shown to be effective at inhibiting the binding of EGF and TGF-α the receptor. (Modjtahedi, H., et al., Int. J. Cancer 75 (1998) 310-316). The murine monoclonal antibody 425 is another MAb that was raised against the human A431 carcinoma cell line and was found to bind to a polypeptide epitope on the external domain of the human epidermal growth factor receptor. (Murthy, U., et al., Arch. Biochem. Biophys. 252 (2) (1987) 549-560. A potential problem with the use of murine antibodies in therapeutic treatments is that non-human monoclonal antibodies can be recognized by the human host as a foreign protein; therefore, repeated injections of such foreign antibodies can lead to the induction of immune responses leading to harmful hypersensitivity reactions. For murine-based monoclonal antibodies, this is often referred to as a Human Anti-Mouse Antibody response, or “HAMA” response, or a Human Anti-Rat Antibody, or “HARA” response. Additionally, these “foreign” antibodies can be attacked by the immune system of the host such that they are, in effect, neutralized before they reach their target site. Furthermore, non-human monoclonal antibodies (e.g., murine monoclonal antibodies) typically lack human effector functionality, i.e., they are unable to, inter alia, mediate complement dependent lysis or lyse human target cells through antibody dependent cellular toxicity or Fc-receptor mediated phagocytosis.

Chimeric antibodies comprising portions of antibodies from two or more different species (e.g., mouse and human) have been developed as an alternative to “conjugated” antibodies. For example, U.S. Pat. No. 5,891,996 (Mateo de Acosta del Rio, C. M., et al.) discusses a mouse/human chimeric antibody, R3, directed against EGFR, and U.S. Pat. No. 5,558,864 discusses generation of chimeric and humanized forms of the murine anti-EGFR MAb 425. Also, IMC-C225 (Erbitux®; ImClone) is a chimeric mouse/human anti-EGFR monoclonal antibody (based on mouse M225 monoclonal antibody, which resulted in HAMA responses in human clinical trials) that has been reported to demonstrate antitumor efficacy in various human xenograft models. (Herbst, R. S., and Shin, D. M., Cancer 94 (2002) 1593-1611). The efficacy of IMC-C225 has been attributed to several mechanisms, including inhibition of cell events regulated by EGFR signaling pathways, and possibly by increased antibody-dependent cellular toxicity (ADCC) activity (Herbst, R. S., and Shin, D. M., Cancer 94 (2002) 1593-1611). IMC-C225 was also used in clinical trials, including in combination with radiotherapy and chemotherapy (Herbst, R. S., and Shin, D. M., Cancer 94 (2002) 1593-1611). Recently, Abgenix, Inc. (Fremont, Calif.) developed ABX-EGF for cancer therapy. ABX-EGF is a fully human anti-EGFR monoclonal antibody. (Yang, X. D., et al., Crit. Rev. Oncol./Hematol. 38 (2001) 17-23).

WO 2006/082515 refers to afocusylated humanized anti-EGFR monoclonal antibodies derived from the rat monoclonal antibody ICR62.

### IGF-1R and IGF-1R Antibodies

Insulin-like growth factor I receptor (also known as IGF-1R or IGF-IR, SwissProt database entry P08069, CD 221 antigen) belongs to the family of transmembrane protein tyrosine kinases (LeRoith, D., et al., Endocrin. Rev. 16 (1995) 143-163; and Adams, T. E., et al., Cell. Mol. Life. Sci. 57 (2000) 1050-1063). IGF-IR binds IGF-I with high affinity and initiates the physiological response to this ligand in vivo. IGF-IR also binds to IGF-II, however with slightly lower affinity. IGF-IR overexpression promotes the neoplastic transformation of cells and there exists evidence that IGF-IR is involved in malignant transformation of cells and is therefore a useful target for the development of therapeutic agents for the treatment of cancer (Adams, T. E., et al., Cell. Mol. Life. Sci. 57 (2000) 1050-1063).

### IGF-1R Antibodies

Antibodies against IGF IR are well-known in the state of the art and investigated for their antitumor effects in vitro and in vivo (Benini, S., et al., Clin. Cancer Res. 7 (2001) 1790-1797; Scotlandi, K., et al., Cancer Gene Ther. 9 (2002) 296-307; Scotlandi, K., et al., Int. J. Cancer 101 (2002) 11-16; Brunetti, A., et al., Biochem. Biophys. Res. Commun. 165 (1989) 212-218; Prigent, S. A., et al., J. Biol. Chem. 265 (1990) 9970-9977; Li, S. L., et al., Cancer Immunol. Immunother. 49 (2000) 243-252; Pessino, A., et al., Biochem. Biophys. Res. Commun. 162 (1989) 1236-1243; Surinya, K. H., et al., J. Biol. Chem. 277 (2002) 16718-16725; Soos, M. A., et al., J. Biol. Chem., 267 (1992) 12955-12963; Soos, M. A., et al., Proc. Natl. Acad. Sci. USA 86 (1989) 5217-5221; O'Brien, R. M., et al., EMBO J. 6 (1987) 4003-4010; Taylor, R., et al., Biochem. J. 242 (1987) 123-129; Soos, M. A., et al., Biochem. J. 235 (1986) 199-208; Li, S. L., et al., Biochem. Biophys. Res. Commun. 196 (1993) 92-98; Delafontaine, P., et al., J. Mol. Cell. Cardiol. 26 (1994) 1659-1673; Kull, F. C. Jr., et al. J. Biol. Chem. 258 (1983) 6561-6566; Morgan, D. O., and Roth, R. A., Biochemistry 25 (1986) 1364-1371; Forsayeth, J. R., et al., Proc. Natl. Acad. Sci. USA 84 (1987) 3448-3451; Schaefer, E. M., et al., J. Biol. Chem. 265 (1990) 13248-13253; Gustafson, T. A., and Rutter, W. J., J. Biol. Chem. 265 (1990) 18663-18667; Hoyne, P. A., et al., FEBS Lett. 469 (2000) 57-60; Tulloch, P. A., et al., J. Struct. Biol. 125 (1999) 11-18; Rohlik, Q. T., et al., Biochem. Biophys. Res. Comm. 149 (1987) 276-281; and Kalebic, T., et al., Cancer Res. 54 (1994) 5531-5534; Adams, T. E., et al., Cell. Mol. Life. Sci. 57 (2000) 1050-1063; Dricu, A., et al., Glycobiology 9 (1999) 571-579; Kanter-Lewensohn, L., et al., Melanoma Res. 8 (1998) 389-397; Li, S. L., et al., Cancer Immunol. Immunother. 49 (2000) 243-252). Antibodies against IGF-IR are also described in a lot of further publications, e.g., Arteaga, C. L., et al., Breast Cancer Res. Treatment 22 (1992) 101-106; and Hailey, J., et al., Mol. Cancer. Ther. 1 (2002) 1349-1353. Examples of human antibodies against IGF-IR are described in WO 02/053596. US 2005/0008642A1 describes in detail anti-IGF-1R antibodies, especially the human anti-IGF-1R antibodies <IGF-1R> HUMAB-Clone 18 (Deposition No. DSM ACC 2587) and <IGF-1R> HUMAB-Clone 22 (Deposition No. DSM ACC 2594). WO 2008/077546 describes the glycoengineered afocusylated human anti-IGF-1R antibodies <IGF-1R> HUMAB-Clone 18 (Deposition No. DSM ACC 2587) and <IGF-1R> HUMAB-Clone 22 (Deposition No. DSM ACC 2594) which a shows increased ADCC.

### Cytokines:

### Properties of Cytokines

Cytokines are small secreted proteins which mediate and regulate immunity, inflammation, and hematopoiesis. They must be produced de novo in response to an immune stimulus. They generally (although not always) act over short distances and short time spans and at very low concentration. They act by binding to specific membrane receptors, which then signal the cell via second messengers, often tyrosine kinases, to alter its behavior (gene expression). Responses to cytokines include increasing or decreasing expression of membrane proteins (including cytokine receptors), proliferation, and secretion of effector molecules.

Cytokine is a general name; other names include lymphokine (cytokines made by lymphocytes), monokine (cytokines made by monocytes), chemokine (cytokines with chemotactic activities), and interleukin (cytokines made by one leukocyte and acting on other leukocytes). Cytokines may act on the cells that secrete them (autocrine action), on nearby cells (paracrine action), or in some instances on distant cells (endocrine action).

It is common for different cell types to secrete the same cytokine or for a single cytokine to act on several different cell types (pleiotropy). Cytokines are redundant in their activity, meaning similar functions can be stimulated by different cytokines. Cytokines are often produced in a cascade, as one cytokine stimulates its target cells to make additional cytokines Cytokines can also act synergistically (two or more cytokines acting together) or antagonistically (cytokines causing opposing activities).

Their short half life, low plasma concentrations, pleiotropy, and redundancy all complicated the isolation and characterization of cytokines Searches for new cytokines is now often conducted at the DNA level, identifying genes similar to known cytokine genes.

### Cytokine Activities

Cytokine activities are characterized using recombinant cytokines and purified cell populations in vitro, or with knock-out mice for individual cytokine genes to characterize cytokine functions in vivo. Cytokines are made by many cell populations, but the predominant producers are helper T cells (Th) and macrophages.

Cytokines stimulate immune cell proliferation and differentiation. The group of Granulocyte Monocyte Colony-Stimulating Factor (GM-CSF), Interleukin-3 (IL-3) and Macrophage colony stimulating factor (M-CSF) differentiate monocytes or pericytes.

Granulocyte Monocyte Colony-Stimulating Factor (GM-CSF) is a cytokine that functions as a white blood cell growth factor. GM-CSF stimulates stem cells to produce granulocytes (neutrophils, eosinophils, and basophils) and monocytes. Monocytes exit the circulation and migrate into tissue, whereupon they mature into macrophages. It is thus part of the immune/inflammatory cascade, by which activation of a small number of macrophages can rapidly lead to an increase in their numbers, a process crucial for fighting infection. The active form of the protein is found extracellularly as a homodimer. (Wong, G. G., et al., Science 228 (1985) 810-5; Lee, F., et al., Proc. Natl. Acad. Sci. U.S.A. 82 (1985) 4360-4; Cantrell, M. A., et al, Proc. Natl. Acad. Sci. U.S.A. 82 (1985) 6250-4).

GM-CSF is used as a medication to stimulate the production of white blood cells following chemotherapy. It has also recently been evaluated in clinical trials for its potential as a vaccine adjuvant in HIV-infected patients. The preliminary results have been promising but GM-CSF is not presently FDA-approved for this purpose. GM-CSF is also known as molgramostim or, when the protein is expressed in yeast cells, sargramostim (Leukine) Leukine™ is the trade name of sargramostim manufactured by Berlex Laboratories, a subsidiary of Schering AG. Its use was approved by U.S. Food and Drug Administration for acceleration of white blood cell recovery following autologous bone marrow transplantation in patients with non-Hodgkin's lymphoma, acute lymphocytic leukemia, or Hodgkin's disease in March 1991. In November 1996, the FDA also approved sargramostim for treatment of fungal infections and replenishment of white blood cells following chemotherapy.

Interleukin-3 (IL-3) is an interleukin, a type of biological signal (cytokine) that can improve the body's natural response to disease as part of the immune system. It acts by binding to the Interleukin-3 receptor.

IL-3 stimulates the differentiation of multipotent hematopoietic stem cells (pluripotent) into myeloid progenitor cells (as opposed to lymphoid progenitor cells where differentiation is stimulated by IL-7) as well as stimulates proliferation of all cells in the myeloid lineage (erythrocytes, thrombocytes, granulocytes, monocytes, and dendritic cells). It is secreted by activated T cells to support growth and differentiation of T cells from the bone marrow in an immune response. The human IL-3 gene encodes a protein 152 amino acids long, and the naturally occurring IL-3 is glycosylated. The human IL-3 gene is located on chromosome 5, only 9 kilobases from the GM-CSF gene, and its function is quite similar to GM-CSF (Yang, Y. C., et al., Cell 47 (1986) 3-10); Urdal, D. L., et al., Ann. N.Y. Acad. Sci. 554 (1989) 167-76; Wagemaker, G., et al., Biotherapy (Dordrecht, Netherlands) 2 (1990) 337-45; Kitamura, T., et al., Cell 66 (1991) 1165-74).

Macrophage colony stimulating factor (M-CSF) is a secreted cytokine which influences hemopoietic stem cells to differentiate into macrophages or other related cell types. Eukaryotic cells also produce M-CSF in order to combat intercellular viral infection. M-CSF binds to the Colony stimulating factor 1 receptor. The active form of the protein is found extracellularly as a disulfide-linked homodimer, and is thought to be produced by proteolytic cleavage of membrane-bound precursors (Kawasaki, E. S., et al., Science 230 (1985) 291-6; Wong, G. G., et al., Science 235 (1987) 1504-8; Ladner, M. B., et al., EMBO J. 6 (1987) 2693-8; Sherr, C. J., et al., Cell 41 (1985) 665-76).

### SUMMARY OF THE INVENTION

The invention comprises a method for the treatment of cancer, comprising administering to a patient in need of such treatment (i) an afucosylated antibody with an amount of fucose is 60% or less; and (ii) one or more cytokines selected from the group consisting of human GM-CSF, human M-CSF and human IL-3.

Preferably said afucosylated antibody is a anti-CD20 antibody, preferably a humanized B-Ly1 antibody, and said cancer is a CD20 expressing cancer, preferably a B-Cell Non-Hodgkin's lymphoma (NHL).

Preferably said afucosylated antibody is an anti-EGFR antibody (preferably a humanized ICR62 antibody) and said cancer is an EGFR-expressing cancer.

Preferably said afucosylated antibody is an anti-IGF-1R antibody (preferably a human HUMAB-Clone 18) and said cancer is an IGF-1R-expressing cancer.

One embodiment of the invention is characterized in that the cancer is a monocytes/pericytes-infiltrated cancer.

One embodiment of the invention is characterized in that as cytokine only GM-CSF is co-administered in said combination treatment.

One embodiment of the invention is characterized in that as cytokine only M-CSF is co-administered in said combination treatment.

One embodiment of the invention is characterized in that as cytokine only IL-3 is co-administered in said combination treatment.

One embodiment of the invention is characterized in that as cytokines only GM-CSF and IL-3 are co-administered in said combination treatment.

One embodiment of the invention is characterized in that the cytokines human GM-CSF, human M-CSF and/or human IL-3 are co-administered in said combination treatment.

One embodiment of the invention is characterized in that the afocusylated antibody shows an increased ADCC.

One embodiment of the invention is a composition comprising an afucosylated antibody specifically binding to a tumor antigen and one or more cytokines selected from human GM-CSF, human M-CSF and/or human IL-3 for the treatment of cancer.

The combination treatment of afocusylated, glycoengineered anti-tumor antigen antibodies in combination with the cytokines GM-CSF, M-CSF and/or IL-3 shows enhanced antitumor inhibitory activity compared to a combination of the corresponding non-afocusylated, non-glycoengineered antibodies with these cytokines GM-CSF, M-CSF and/or IL-3. The combination treatment mediates antitumor efficacy via monocytes/pericytes which are differentiated into macrophages by these cytokines GM-CSF, M-CSF and/or IL-3 and is especially valuable for the treatment of cancers which are infiltrated by monocytes/pericytes.

### DETAILED DESCRIPTION OF THE INVENTION

The invention comprises a method for the treatment of cancer, comprising administering to a patient in need of such treatment (i) an afucosylated antibody of IgG1 or IgG3 isotype (preferably of IgG1 isotype) specifically binding to a tumor antigen with an amount of fucose is 60% or less of the total amount of oligosaccharides (sugars) at Asn297; and (ii) one or more cytokines selected from the group consisting of human GM-CSF, human M-CSF and human IL-3, wherein the cancer expresses said tumor antigen.

In one embodiment the amount of fucose is between 20% and 60% of the total amount of oligosaccharides (sugars) at Asn297.

The term “antibody” encompasses the various forms of antibodies including but not being limited to whole antibodies, human antibodies, humanized antibodies and genetically engineered antibodies like monoclonal antibodies, chimeric antibodies or recombinant antibodies as well as fragments of such antibodies as long as the characteristic properties according to the invention are retained. The terms “monoclonal antibody” or “monoclonal antibody composition” as used herein refer to a preparation of antibody molecules of a single amino acid composition. Accordingly, the term “human monoclonal antibody” refers to antibodies displaying a single binding specificity which have variable and constant regions derived from human germline immunoglobulin sequences. In one embodiment, the human monoclonal antibodies are produced by a hybridoma which includes a B cell obtained from a transgenic non-human animal, e.g. a transgenic mouse, having a genome comprising a human heavy chain transgene and a light human chain transgene fused to an immortalized cell.

The term “chimeric antibody” refers to a monoclonal antibody comprising a variable region, i.e., binding region, from one source or species and at least a portion of a constant region derived from a different source or species, usually prepared by recombinant DNA techniques. Chimeric antibodies comprising a murine variable region and a human constant region are especially preferred. Such murine/human chimeric antibodies are the product of expressed immunoglobulin genes comprising DNA segments encoding murine immunoglobulin variable regions and DNA segments encoding human immunoglobulin constant regions. Other forms of “chimeric antibodies” encompassed by the present invention are those in which the class or subclass has been modified or changed from that of the original antibody. Such “chimeric” antibodies are also referred to as “class-switched antibodies.” Methods for producing chimeric antibodies involve conventional recombinant DNA and gene transfection techniques now well known in the art. See, e.g., Morrison, S. L., et al., Proc. Natl. Acad. Sci. USA 81 (1984) 6851-6855; U.S. Pat. No. 5,202,238 and U.S. Pat. No. 5,204,244.

The term “humanized antibody” refers to antibodies in which the framework or “complementarity determining regions” (CDR) have been modified to comprise the CDR of an immunoglobulin of different specificity as compared to that of the parent immunoglobulin. In a preferred embodiment, a murine CDR is grafted into the framework region of a human antibody to prepare the “humanized antibody.” See, e.g., Riechmann, L., et al., Nature 332 (1988) 323-327; and Neuberger, M. S., et al., Nature 314 (1985) 268-270. Particularly preferred CDRs correspond to those representing sequences recognizing the antigens noted above for chimeric and bifunctional antibodies.

The term “human antibody”, as used herein, is intended to include antibodies having variable and constant regions derived from human germline immunoglobulin sequences. Human antibodies are well-known in the state of the art (van Dijk, M. A., and van de Winkel, J. G., Curr. Opin. Chem. Biol 5 (2001) 368-374). Based on such technology, human antibodies against a great variety of targets can be produced. Examples of human antibodies are for example described in Kellermann, S. A., et al., Curr Opin Biotechnol. 13 (2002) 593-597.

The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions derived from human germline immunoglobulin sequences in a rearranged form. The recombinant human antibodies according to the invention have been subjected to in vivo somatic hypermutation. Thus, the amino acid sequences of the VH and VL regions of the recombinant antibodies are sequences that, while derived from and related to human germline VH and VL sequences, may not naturally exist within the human antibody germline repertoire in vivo.

As used herein, the term “binding” or “specifically binding” refers to the binding of the antibody to an epitope of the tumor antigen in an in vitro assay, preferably in an plasmon resonance assay (BIAcore, GE-Healthcare Uppsala, Sweden) with purified wild-type antigen. The affinity of the binding is defined by the terms ka (rate constant for the association of the antibody from the antibody/antigen complex), k_{D} (dissociation constant), and K_{D} (k_{D}/ka). Binding or specifically binding means a binding affinity (K_{D}) of 10⁻⁸ mol/l or less, preferably 10⁻⁹ M to 10⁻¹³ mol/l. Thus, an afocusylated antibody according to the invention is specifically binding to the tumor antigen with a binding affinity (K_{D}) of 10⁻⁸ mol/l or less, preferably 10⁻⁹ M to 10⁻¹³ mol/l.

The term “nucleic acid molecule”, as used herein, is intended to include DNA molecules and RNA molecules. A nucleic acid molecule may be single-stranded or double-stranded, but preferably is double-stranded DNA.

The “constant domains” are not involved directly in binding the antibody to an antigen but are involved in the effector functions (ADCC, complement binding, and CDC).

The “variable region” (variable region of a light chain (VL), variable region of a heavy chain (VH)) as used herein denotes each of the pair of light and heavy chains which is involved directly in binding the antibody to the antigen. The domains of variable human light and heavy chains have the same general structure and each domain comprises four framework (FR) regions whose sequences are widely conserved, connected by three “hypervariable regions” (or complementarity determining regions, CDRs). The framework regions adopt a b-sheet conformation and the CDRs may form loops connecting the b-sheet structure. The CDRs in each chain are held in their three-dimensional structure by the framework regions and form together with the CDRs from the other chain the antigen binding site.

The terms “hypervariable region” or “antigen-binding portion of an antibody” when used herein refer to the amino acid residues of an antibody which are responsible for antigen-binding. The hypervariable region comprises amino acid residues from the “complementarity determining regions” or “CDRs”. “Framework” or “FR” regions are those variable domain regions other than the hypervariable region residues as herein defined. Therefore, the light and heavy chains of an antibody comprise from N- to C-terminus the domains FR1, CDR1, FR2, CDR2, FR3, CDR3, and FR4. Especially, CDR3 of the heavy chain is the region which contributes most to antigen binding. CDR and FR regions are determined according to the standard definition of Kabat, et al., Sequences of Proteins of Immunological Interest, 5th Ed. Public Health Service, National Institutes of Health, Bethesda, Md. (1991)) and/or those residues from a “hypervariable loop”.

Human “GM-CSF” (granulocyte-macrophage colony stimulating factor, GMCSF, colony stimulating factor 2 (granulocyte-macrophage), colony stimulating factor 2, CSF2, variants of SEQ ID NO: 21-22 with >98.5% identity of the amino acid sequences) is a cytokine that controls the production, differentiation, and function of granulocytes and macrophages. The active form of the protein is found extracellularly as a homodimer (Wong, G. G., et al., Science 228 (1985) 810-5; Lee, F., et al., Proc. Natl. Acad. Sci. U.S.A. 82 (1985) 4360-4; Cantrell, M. A., et al., Proc. Natl. Acad. Sci. U.S.A. 82 (1985) 6250-4). GM-CSF is also known as molgramostim or, when the protein is expressed in yeast cells, sargramostim (Leukine) Leukine™ is the trade name of sargramostim manufactured by Berlex Laboratories, a subsidiary of Schering AG. Its use was approved by U.S. Food and Drug Administration. The term “GM-CSF” according to the invention refers to human granulocyte-macrophage colony stimulating factor with an amino acid sequence of >98.5% identity to SEQ ID NO: 21. Preferably GM-CSF has an amino acid sequence of SEQ ID NO: 21.

Human “IL-3” (interleukin 3, MCGF (mast-cell growth factor), P-cell stimulating factor, hematopoietic growth factor, multilineage-colony-stimulating factor, variants of SEQ ID NO: 23-25 with >98.5% identity of the amino acid sequences) is a a potent growth promoting cytokine. This cytokine is capable of supporting the proliferation of a broad range of hematopoietic cell types. It is involved in a variety of cell activities such as cell growth, differentiation and apoptosis Yang, Y. C., et al., Cell 47 (1986) 3-10; Urdal, D. L., et al., Ann. N.Y. Acad. Sci. 554 (1989) 167-76; Wagemaker, G., et al., Biotherapy (Dordrecht, Netherlands) 2 (1990) 337-45; Kitamura, T., et al., Cell 66 (1991) 1165-74. Interleukin-3 (IL-3) is an interleukin, a type of biological signal (cytokine) that can improve the body's natural response to disease as part of the immune system. It acts by binding to the Interleukin-3 receptor. IL-3 stimulates the differentiation of multipotent hematopoietic stem cells (pluripotent) into myeloid progenitor cells (as opposed to lymphoid progenitor cells where differentiation is stimulated by IL-7) as well as stimulates proliferation of all cells in the myeloid lineage (erythrocytes, thrombocytes, granulocytes, monocytes, and dendritic cells). It is secreted by activated T cells to support growth and differentiation of T cells from the bone marrow in an immune response. The human IL-3 gene encodes a protein 152 amino acids long, and the naturally occurring IL-3 is glycosylated. The human IL-3 gene is located on chromosome 5, only 9 kilobases from the GM-CSF gene, and its function is quite similar to GM-CSF. The term “IL-3” according to the invention refers to human interleukin 3 with an amino acid sequence of >98.5% identity to SEQ ID NO: 23. Preferably IL-3 has an amino acid sequence of SEQ ID NO: 23.

Human “M-CSF” (macrophage colony stimulating factor, MCSF, colony stimulating factor 1 (macrophage), colony stimulating factor 1, CSF1; variants of SEQ ID NO: 26-27 with >98.5% identity of the amino acid sequences SEQ ID NO: 26) is a cytokine that controls the production, differentiation, and function of macrophages. The active form of the protein is found extracellularly as a disulfide-linked homodimer, and is thought to be produced by proteolytic cleavage of membrane-bound precursors. The encoded protein may be involved in development of the placenta. Four transcript variants encoding three different isoforms have been found for this gene (Kawasaki, E. S., et al., Science 230 (1985) 291-6; Wong, G. G., et al., Science 235: (1987) 1504-8; Ladner, M. B., et al., EMBO J. 6 (1987) 2693-8). The term “M-CSF” according to the invention refers to human macrophage colony stimulating factor with an amino acid sequence of >99.5% identity to SEQ ID NO: 26. Preferably M-CSF has an amino acid sequence of SEQ ID NO:26.

The term “afucosylated antibody” refers to an antibody of IgG1 or IgG3 isotype (preferably of IgG1 isotype) with an altered pattern of glycosylation in the Fc region at Asn297 having a reduced level of fucose residues. Glycosylation of human IgG1 or IgG3 occurs at Asn297 as core fucosylated bianntennary complex oligosaccharide glycosylation terminated with up to 2 Gal residues. These structures are designated as G0, G1 (α1,6 or α1,3) or G2 glycan residues, depending from the amount of terminal Gal residues (Raju, T. S., BioProcess Int. 1 (2003) 44-53). CHO type glycosylation of antibody Fc parts is e.g. described by Routier, F. H., Glycoconjugate J. 14 (1997) 201-207. Antibodies which are recombinantely expressed in non glycomodified CHO host cells usually are fucosylated at Asn297 in an amount of at least 85%.

Thus an afucosylated antibody according to the invention means an antibody of IgG1 or IgG3 isotype (preferably of IgG1 isotype) wherein the amount of fucose is 60% or less of the total amount of oligosaccharides (sugars) at Asn297 (which means that at least 40% or more of the oligosaccharides of the Fc region at Asn297 are afucosylated). In one embodiment the amount of fucose is between 20% and 60% of the oligosaccharides of the Fc region at Asn297. In one embodiment the amount of fucose is between 40% and 60% of the oligosaccharides of the Fc region at Asn297. In another embodiment the amount of fucose is 50% or less, and in still another embodiment the amount of fucose is 30% or less of the oligosaccharides of the Fc region at Asn297. According to the invention “amount of fucose” means the amount of said oligosaccharide (fucose) within the oligosaccharide (sugar) chain at Asn297, related to the sum of all oligosaccharides (sugars) attached to Asn 297 (e.g. complex, hybrid and high mannose structures) measured by MALDI-TOF mass spectrometry and calculated as average value (for a detailed procedure to determine the amount of fucose, see Example 8).

Furthermore the oligosaccharides of the Fc region are preferably bisected. The afucosylated antibody according to the invention can be expressed in a glycomodified host cell engineered to express at least one nucleic acid encoding a polypeptide having GnTIII activity in an amount sufficient to partially fucosylate the oligosaccharides in the Fc region. In one embodiment, the polypeptide having GnTIII activity is a fusion polypeptide. Alternatively α1,6-fucosyltransferase activity of the host cell can be decreased or eliminated according to U.S. Pat. No. 6,946,292 to generate glycomodified host cells. The amount of antibody fucosylation can be predetermined e.g. either by fermentation conditions (e.g. fermentation time) or by combination of at least two antibodies with different fucosylation amount. Such afucosylated antibodies and respective glycoengineering methods are described in WO 2005/044859, WO 2004/065540, WO2007/031875, Umana, P., et al., Nature Biotechnol. 17 (1999) 176-180, WO 99/154342, WO 2005/018572, WO 2006/116260, WO 2006/114700, WO 2005/011735, WO 2005/027966, WO 97/028267, US 2006/0134709, US 2005/0054048, US 2005/0152894, WO 2003/035835, WO 2000/061739. These glycoengineered antibodies have an increased ADCC. Other glycoengineering methods yielding afocusylated antibodies according to the invention are described e.g. in Niwa, R., et al., J. Immunol. Methods 306 (2005) 151-160; Shinkawa, T. et al, J Biol Chem, 278 (2003) 3466-3473; WO 03/055993 or US 2005/0249722.

One embodiment of the invention is characterized in that the afocusylated antibody shows an increased ADCC (compared to the corresponding non-afocusylated parent antibody). In one embodiment the afocusylated antibody has an increased ADCC compared to the corresponding non-afocusylated parent antibody of at least 50% (at 10 ng/ml antibody concentration and a effector cells/tumor cell E:T ratio of 25:1 with freshly isolated PBMC as Effector cells and suitable antigen-expressing tumor cells (e.g. H322M for IGF1-R, Raji for CD20 and A549 for EGFR).

The afucosylated antibodies according to the invention, as e.g. anti-CD20 antibodies, anti-EGFR antibodies or anti-IGF-1R antibodies, have an increased antibody dependent cellular cytotoxicity (ADCC).

By “afucosylated antibodies (e.g. anti-CD20 antibodies, anti-EGFR antibodies or anti-IGF-1R antibodies) with increased antibody dependent cellular cytotoxicity (ADCC)” is meant an afucosylated antibody (e.g. anti-CD20 antibody, anti-EGFR antibody or anti-IGF-1R antibody), as that term is defined herein, having increased ADCC as determined by any suitable method known to those of ordinary skill in the art.

One accepted in vitro ADCC assay to determine the increased ADCC of the afocusylated antibody compared to the corresponding wild type parent antibody is described in WO 2005/044859:

1) the assay uses target cells that are known to express the target antigen recognized by the antigen-binding region of the antibody;
2) the assay uses human peripheral blood mononuclear cells (PBMCs), isolated from blood of a randomly chosen healthy donor, as effector cells;
3) the assay is carried out according to following protocol:
i) the PBMCs are isolated using standard density centrifugation procedures and are suspended at 5×10⁶ cells/ml in RPMI cell culture medium;
ii) the target cells are grown by standard tissue culture methods, harvested from the exponential growth phase with a viability higher than 90%, washed in RPMI cell culture medium, labeled with 100 micro-Curies of ⁵¹Cr, washed twice with cell culture medium, and resuspended in cell culture medium at a density of 10⁵ cells/ml;
iii) 100 microliters of the final target cell suspension above are transferred to each well of a 96-well microtiter plate;
iv) the antibody is serially-diluted from 4000 ng/ml to 0.04 ng/ml in cell culture medium and 50 microliters of the resulting antibody solutions are added to the target cells in the 96-well microtiter plate, testing in triplicate various antibody concentrations covering the whole concentration range above;
v) for the maximum release (MR) controls, 3 additional wells in the plate containing the labeled target cells, receive 50 microliters of a 2% (VN) aqueous solution of non-ionic detergent (Nonidet, Sigma, St. Louis), instead of the antibody solution (point iv above);
vi) for the spontaneous release (SR) controls, 3 additional wells in the plate containing the labeled target cells, receive 50 microliters of RPMI cell culture medium instead of the antibody solution (point iv above);
vii) the 96-well microtiter plate is then centrifuged at 50×g for 1 minute and incubated for 1 hour at 4° C.;
viii) 50 microliters of the PBMC suspension (point i above) are added to each well to yield an effector:target cell ratio of 25:1 and the plates are placed in an incubator under 5% CO2 atmosphere at 37 C for 4 hours;
ix) the cell-free supernatant from each well is harvested and the experimentally released radioactivity (ER) is quantified using a gamma counter;
x) the percentage of specific lysis is calculated for each antibody concentration according to the formula (ER−MR)/(MR−SR)×100, where ER is the average radioactivity quantified (see point ix above) for that antibody concentration, MR is the average radioactivity quantified (see point ix above) for the MR controls (see point V above), and SR is the average radioactivity quantified (see point ix above) for the SR controls (see point vi above);
4) “increased ADCC” is defined as either an increase in the maximum percentage of specific lysis observed within the antibody concentration range tested above, and/or a reduction in the concentration of antibody required to achieve one half of the maximum percentage of specific lysis observed within the antibody concentration range tested above. In a preferred embodiment increased ADCC is defined as an increase in the percentage of specific lysis observed at 10 ng/ml antibody concentration and a effector cells/tumor cell E:T ratio of 25:1 with freshly isolated PBMC as Effector cells and suitable antigen-expressing tumor cells (e.g. H322M for IGF1-R, Raji for CD20 and A549 for EGFR after 4 h.

The increase in ADCC is relative to the ADCC, measured with the above assay, mediated by the same antibody, produced by the same type of host cells, using the same standard production, purification, formulation and storage methods, which are known to those skilled in the art, but that has not been produced by host cells engineered to overexpress GnTIII.

Said “increased ADCC” can be obtained by glycoengineering of said antibodies, that means enhance said natural, cell-mediated effector functions of monoclonal antibodies by engineering their oligosaccharide component as described in Umana, P., et al., Nature Biotechnol. 17 (1999) 176-180 and U.S. Pat. No. 6,602,684. The amount of fucose in such glycoengineered antibodies is 60% or lower, whereas the amount of fucose int eh correspongin wild type parent antibodies (in which the glycostructure is not engineered) is usually 85% or higher.

The term “complement-dependent cytotoxicity (CDC)” refers to lysis of human tumor target cells by the antibody according to the invention in the presence of complement. CDC is measured preferably by the treatment of a preparation of CD20 expressing cells with an anti-CD20 antibody according to the invention in the presence of complement. CDC is found if the antibody induces at a concentration of 100 nM the lysis (cell death) of 20% or more of the tumor cells after 4 hours. The assay is performed preferably with ⁵¹Cr or Eu labeled tumor cells and measurement of released ⁵¹Cr or Eu. Controls include the incubation of the tumor target cells with complement but without the antibody.

A “tumor antigen,” as used herein, refers to a tumor antigen of human origin and includes the meaning known in the art, which includes any molecule expressed on (or associated with the development of) a tumor cell that is known or thought to contribute to a tumorigenic characteristic of the tumor cell. Numerous tumor antigens are known in the art. Whether a molecule is a tumor antigen can also be determined according to techniques and assays well known to those skilled in the art, such as for example clonogenic assays, transformation assays, in vitro or in vivo tumor formation assays, gel migration assays, gene knockout analysis, etc. Preferably the term “tumor antigen” when used herein refers to a human transmembrane protein i.e., a cell membrane proteins which is anchored in the lipid bilayer of cells. The human transmembrane protein will generally comprise an “extracellular domain” as used herein, which may bind a ligand; a lipophilic transmembrane domain, a conserved intracellular domain tyrosine kinase domain, and a carboxyl-terminal signaling domain harboring several tyrosine residues which can be phosphorylated. The tumor antigen include molecules such as EGFR, HER2/neu, HER3, HER4, Ep-CAM, CEA, TRAIL, TRAIL-receptor 1, TRAIL-receptor 2, lymphotoxin-beta receptor, CCR4, CD19, CD20, CD22, CD28, CD33, CD40, CD44, CD80, CSF-1R, CTLA-4, fibroblast activation protein (FAP), hepsin, melanoma-associated chondroitin sulfate proteoglycan (MCSP), prostate-specific membrane antigen (PSMA), CDCP1, VEGF receptor 1, VEGF receptor 2, IGF1-R, TSLP-R, TIE-1, TIE-2, TNF-alpha, TNF like weak inducer of apoptosis (TWEAK), IL-1R, preferably MCSP, EGFR, CEA, CD20, or IGF1-R, more preferably CD20, IGF1-R or EGFR, still more preferably CD20 or EGFR. Therefore said afucosylated antibody according to the invention is preferably an anti-CD20, -IGF1-R or -EGFR antibody.

Thus one aspect of the invention is the use of an afucosylated antibody of IgG1 or IgG3 isotype (preferably of IgG1 isotype) specifically binding to a tumor antigen with an amount of fucose of 60% or less of the total amount of oligosaccharides (sugars) at Asn297, for the manufacture of a medicament for the treatment of cancer in combination with one or more cytokines selected from the group of GM-CSF, M-CSF and IL-3, wherein the tumor antigen is selected from EGFR, HER2/neu, HER3, HER4, Ep-CAM, CEA, TRAIL, TRAIL-receptor 1, TRAIL-receptor 2, lymphotoxin-beta receptor, CCR4, CD19, CD20, CD22, CD28, CD33, CD40, CD44, CD80, CSF-1R, CTLA-4, fibroblast activation protein (FAP), hepsin, melanoma-associated chondroitin sulfate proteoglycan (MCSP), prostate-specific membrane antigen (PSMA), CDCP1, VEGF receptor 1, VEGF receptor 2, IGF1-R, TSLP-R, TIE-1, TIE-2, TNF-alpha, TNF like weak inducer of apoptosis (TWEAK), IL-1R, preferably from MCSP, EGFR, CEA, CD20, or IGF1-R, more preferably from CD20, IGF1-R or EGFR, still more preferably from CD20 or EGFR. In one embodiment the amount of fucose is between 20% and 60% of the total amount of oligosaccharides (sugars) at Asn297. In another embodiment the amount of fucose is between 40% and 60% of the total amount of oligosaccharides (sugars) at Asn297.

Thus one aspect of the invention is the use of an afucosylated antibody of IgG1 or IgG3 isotype (preferably of IgG1 isotype) specifically binding to a tumor antigen with an amount of fucose of 60% or less of the total amount of oligosaccharides (sugars) at Asn297, for the manufacture of a medicament for the treatment of cancer in combination with one or more cytokines selected from the group of GM-CSF, M-CSF and IL-3, wherein the cancer expresses said tumor antigen which is selected from EGFR, HER2/neu, HER3, HER4, Ep-CAM, CEA, TRAIL, TRAIL-receptor 1, TRAIL-receptor 2, lymphotoxin-beta receptor, CCR4, CD19, CD20, CD22, CD28, CD33, CD40, CD44, CD80, CSF-1R, CTLA-4, fibroblast activation protein (FAP), hepsin, melanoma-associated chondroitin sulfate proteoglycan (MCSP), prostate-specific membrane antigen (PSMA), CDCP1, VEGF receptor 1, VEGF receptor 2, IGF1-R, TSLP-R, TIE-1, TIE-2, TNF-alpha, TNF like weak inducer of apoptosis (TWEAK), IL-1R, preferably from MCSP, EGFR, CEA, CD20, or IGF1-R, more preferably from CD20, IGF1-R or EGFR, still more preferably from CD20 or EGFR. In one embodiment the amount of fucose is between 20% and 60% of the total amount of oligosaccharides (sugars) at Asn297. In another embodiment the amount of fucose is between 40% and 60% of the total amount of oligosaccharides (sugars) at Asn297.

As used herein, the term “binding” or “specifically binding” refers to the binding of the antibody to an epitope of the antigen in an in vitro assay, preferably in an plasmon resonance assay (BIAcore, GE-Healthcare Uppsala, Sweden) with purified wild-type antigen. The affinity of the binding is defined by the terms ka (rate constant for the association of the antibody from the antibody/antigen complex), k_{D} (dissociation constant), and K_{D} (k_{D}/ka). Binding or specifically binding means a binding affinity (K_{D}) of 10⁻⁸ mol/l or less, preferably 10⁻⁹ M to 10⁻¹³ mol/l. Thus, an afocusalyted antibody according to the invention is specifically binding to a tumor antigen for which it is specific with a binding affinity (K_{D}) of 10⁻⁸ mol/l or less, preferably 10⁻⁹ M to 10⁻¹³ mol/l.

“EGFR” as used herein refers to the human epidermal growth factor receptor (also known as HER-1 or Erb-B1, and referred to herein as “EGFR”) is a 170 kDa transmembrane receptor encoded by the c-erbB proto-oncogene, and exhibits intrinsic tyrosine kinase activity (Modjtahedi, H., et al., Br. J. Cancer 73 (1996) 228-235; Herbst, R. S., and Shin, D. M., Cancer 94 (2002) 1593-1611). SwissProt database entry P00533 provides the sequence of EGFR. There are also isoforms and variants of EGFR (e.g., alternative RNA transcripts, truncated versions, polymorphisms, etc.) including but not limited to those identified by Swissprot database entry numbers P00533-1, P00533-2, P00533-3, and P00533-4. EGFR is known to bind ligands including epidermal growth factor (EGF), transforming growth factor-α (TGf-α), amphiregulin, heparin-binding EGF (hb-EGF), betacellulin, and epiregulin (Herbst, R. S., and Shin, D. M., Cancer 94 (2002) 1593-1611; Mendelsohn, J., and Baselga, J., Oncogene 19 (2000) 6550-6565). EGFR regulates numerous cellular processes via tyrosine-kinase mediated signal transduction pathways, including, but not limited to, activation of signal transduction pathways that control cell proliferation, differentiation, cell survival, apoptosis, angiogenesis, mitogenesis, and metastasis (Atalay, G., et al., Ann. Oncology 14 (2003) 1346-1363; Tsao, A. S., and Herbst, R. S., Signal 4 (2003) 4-9; Herbst, R. S., and Shin, D. M., Cancer 94 (2002) 1593-1611; Modjtahedi, H., et al., Br. J. Cancer 73 (1996) 228-235).

The term “afocusylated anti-EGFR antibody” according to the invention is an antibody that binds specifically to human IGF-1R antigen. Examples for anti-EGFR antibodies are described e.g. in WO 2006/082515 which disclose afocusylated humanized anti-EGFR monoclonal antibodies derived from the rat monoclonal antibody ICR62. In a preferred embodiment the afocusylated anti-EGFR antibody refers to afocusylated humanized ICR62 comprising as heavy chain variable domain SEQ ID NO: 30, and as light chain variable domain SEQ ID NO: 31.

“IGF-1R” as used herein refers to the human Insulin-like growth factor I receptor (also known as IGF-1R or IGF-IR, SwissProt database entry P08069, CD 221 antigen) belongs to the family of transmembrane protein tyrosine kinases (LeRoith, D., et al., Endocrin. Rev. 16 (1995) 143-163; and Adams, T. E., et al., Cell. Mol. Life. Sci. 57 (2000) 1050-1063). IGF-IR binds IGF-I with high affinity and initiates the physiological response to this ligand in vivo. IGF-IR also binds to IGF-II, however with slightly lower affinity. IGF-IR overexpression promotes the neoplastic transformation of cells and there exists evidence that IGF-IR is involved in malignant transformation of cells and is therefore a useful target for the development of therapeutic agents for the treatment of cancer (Adams, T. E., et al., Cell. Mol. Life. Sci. 57 (2000) 1050-1063).

The term “anti-IGF-1R antibody” according to the invention is an antibody that binds specifically to human IGF-1R antigen. Examples for anti-IGF1-R antibodies are well-known in the state of the art and investigated for their antitumor effects in vitro and in vivo (Benini, S., et al., Clin. Cancer Res. 7 (2001) 1790-1797; Scotlandi, K., et al., Cancer Gene Ther. 9 (2002) 296-307; Scotlandi, K., et al., Int. J. Cancer 101 (2002) 11-16; Brunetti, A., et al., Biochem. Biophys. Res. Commun. 165 (1989) 212-218; Prigent, S. A., et al., J. Biol. Chem. 265 (1990) 9970-9977; Li, S. L., et al., Cancer Immunol. Immunother. 49 (2000) 243-252; Pessino, A., et al., Biochem. Biophys. Res. Commun. 162 (1989) 1236-1243; Surinya, K. H., et al., J. Biol. Chem. 277 (2002) 16718-16725; Soos, M. A., et al., J. Biol. Chem., 267 (1992) 12955-12963; Soos, M. A., et al., Proc. Natl. Acad. Sci. USA 86 (1989) 5217-5221; O'Brien, R. M., et al., EMBO J. 6 (1987) 4003-4010; Taylor, R., et al., Biochem. J. 242 (1987) 123-129; Soos, M. A., et al., Biochem. J. 235 (1986) 199-208; Li, S. L., et al., Biochem. Biophys. Res. Commun. 196 (1993) 92-98; Delafontaine, P., et al., J. Mol. Cell. Cardiol. 26 (1994) 1659-1673; Kull, F. C. Jr., et al. J. Biol. Chem. 258 (1983) 6561-6566; Morgan, D. O., and Roth, R. A., Biochemistry 25 (1986) 1364-1371; Forsayeth, J. R., et al., Proc. Natl. Acad. Sci. USA 84 (1987) 3448-3451; Schaefer, E. M., et al., J. Biol. Chem. 265 (1990) 13248-13253; Gustafson, T. A., and Rutter, W. J., J. Biol. Chem. 265 (1990) 18663-18667; Hoyne, P. A., et al., FEBS Lett. 469 (2000) 57-60; Tulloch, P. A., et al., J. Struct. Biol. 125 (1999) 11-18; Rohlik, Q. T., et al., Biochem. Biophys. Res. Comm. 149 (1987) 276-281; and Kalebic, T., et al., Cancer Res. 54 (1994) 5531-5534; Adams, T. E., et al., Cell. Mol. Life. Sci. 57 (2000) 1050-1063; Dricu, A., et al., Glycobiology 9 (1999) 571-579; Kanter-Lewensohn, L., et al., Melanoma Res. 8 (1998) 389-397; Li, S. L., et al., Cancer Immunol. Immunother. 49 (2000) 243-252). Antibodies against IGF-IR are also described in a lot of further publications, e.g., Arteaga, C. L., et al., Breast Cancer Res. Treatment 22 (1992) 101-106; and Hailey, J., et al., Mol. Cancer. Ther. 1 (2002) 1349-1353. Examples of human antibodies against IGF-IR are described in WO 02/053596. Further anti-IGF-1R antibodies are described in WO 2003/059951 and WO 2003/100008. US 2005/0008642A1 describes in detail anti-IGF-1R antibodies, especially the human anti-IGF-1R antibodies <IGF-1R> HUMAB-Clone 18 (Deposition No. DSM ACC 2587) and <IGF-1R> HUMAB-Clone 22 (Deposition No. DSM ACC 2594).

WO 2008/077546 describes the glycoengineered, afocusylated human anti-IGF-1R antibodies HUMAB-Clone 18 and HUMAB-Clone 22 which show increased ADCC. In a preferred embodiment of the invention the afocusylated antibody is an anti-IGF1-R antibody, preferably the afocusylated HUMAB-Clone 18 described in WO 2008/077546 comprising as heavy chain variable domain SEQ ID NO: 28, and as light chain variable domains of SEQ ID NO: 29.

“CD20” as used herein refers to the human B-lymphocyte antigen CD20 (also known as CD20, B-lymphocyte surface antigen B1, Leu-16, Bp35, BM5, and LF5; the sequence is characterized by the SwissProt database entry P11836) is a hydrophobic transmembrane protein with a molecular weight of approximately 35 kD located on pre-B and mature B lymphocytes. (Valentine, M. A., et al., J. Biol. Chem. 264(19) (1989 11282-11287; Tedder, T. F., et al, Proc. Natl. Acad. Sci. U.S.A. 85 (1988) 208-12; Stamenkovic, I., et al., J. Exp. Med. 167 (1988) 1975-80; Einfeld, D. A., et al., EMBO J. 7 (1988) 711-7; Tedder, T. F., et al., J. Immunol. 142 (1989) 2560-8). The corresponding human gene is Membrane-spanning 4-domains, subfamily A, member 1, also known as MS4A1. This gene encodes a member of the membrane-spanning 4A gene family. Members of this nascent protein family are characterized by common structural features and similar intron/exon splice boundaries and display unique expression patterns among hematopoietic cells and nonlymphoid tissues. This gene encodes the B-lymphocyte surface molecule which plays a role in the development and differentiation of B-cells into plasma cells. This family member is localized to 11q12, among a cluster of family members. Alternative splicing of this gene results in two transcript variants which encode the same protein.

The terms “CD20” and “CD20 antigen” are used interchangeably herein, and include any variants, isoforms and species homologs of human CD20 which are naturally expressed by cells or are expressed on cells transfected with the CD20 gene. Binding of an antibody of the invention to the CD20 antigen mediate the killing of cells expressing CD20 (e.g., a tumor cell) by inactivating CD20. The killing of the cells expressing CD20 may occur by one or more of the following mechanisms: Cell death/apoptosis induction, ADCC and CDC.

Synonyms of CD20, as recognized in the art, include B-lymphocyte antigen CD20, B-lymphocyte surface antigen B1, Leu-16, Bp35, BM5, and LF5.

The term “anti-CD20 antibody” according to the invention is an antibody that binds specifically to CD20 antigen. Depending on binding properties and biological activities of anti-CD20 antibodies to the CD20 antigen, two types of anti-CD20 antibodies (type I and type II anti-CD20 antibodies) can be distinguished according to Cragg, M. S., et al., Blood 103 (2004) 2738-2743; and Cragg, M. S., et al., Blood 101 (2003) 1045-1052, see Table 2.

TABLE 2
Properties of type I and type II anti-CD20 antibodies

| Type I anti-CD20 antibodies | type II anti-CD20 antibodies |
|---|---|
| type I CD20 epitope | type II CD20 epitope |
| Localize CD20 to lipid rafts | Do not localize CD20 to lipid rafts |
| Increased CDC (if IgG1 isotype) | Decreased CDC (if IgG1 isotype) |
| ADCC activity (if IgG1 isotype) | ADCC activity (if IgG1 isotype) |
| Full binding capacity | Reduced binding capacity |
| Homotypic aggregation | Stronger homotypic aggregation |
| Apoptosis induction upon | Strong cell death induction without |
| cross-linking | cross-linking |

Examples of type II anti-CD20 antibodies include e.g. humanized B-Ly1 antibody IgG1 (a chimeric humanized IgG1 antibody as disclosed in WO 2005/044859), 11B8 IgG1 (as disclosed in WO 2004/035607), and AT80 IgG1. Typically type II anti-CD20 antibodies of the IgG1 isotype show characteristic CDC properties. Type II anti-CD20 antibodies have a decreased CDC (if IgG1 isotype) compared to type I antibodies of the IgG1 isotype.

Examples of type I anti-CD20 antibodies include e.g. rituximab, HI47 IgG3 (ECACC, hybridoma), 2C6 IgG1 (as disclosed in WO 2005/103081), 2F2 IgG1 (as disclosed and WO 2004/035607 and WO 2005/103081) and 2H7 IgG1 (as disclosed in WO 2004/056312).

The afucosylated anti-CD20 antibodies according to the invention is preferably a type II anti-CD20 antibodies, more preferably an afucosylated humanized B-Ly1 antibody as described in WO 2005/044859 and WO 2007/031875.

The “rituximab” antibody (reference antibody; example of a type I anti-CD20 antibody) is a genetically engineered chimeric human gamma 1 murine constant domain containing monoclonal antibody directed against the human CD20 antigen. However this antibody is not glycoengineered and not afocusylates and thus has an amount of fucose of at least 85%. This chimeric antibody contains human gamma 1 constant domains and is identified by the name “C2B8” in U.S. Pat. No. 5,736,137 (Andersen, et. al.) issued on Apr. 17, 1998, assigned to IDEC Pharmaceuticals Corporation. Rituximab is approved for the treatment of patients with relapsed or refracting low-grade or follicular, CD20 positive, B cell non-Hodgkin's lymphoma. In vitro mechanism of action studies have shown that rituximab exhibits human complement-dependent cytotoxicity (CDC) (Reff, M. E., et. al, Blood 83(2) (1994) 435-445). Additionally, it exhibits activity in assays that measure antibody-dependent cellular cytotoxicity (ADCC). The term “humanized B-Ly1 antibody” refers to humanized B-Ly1 antibody as disclosed in WO 2005/044859 and WO 2007/031875, which were obtained from the murine monoclonal anti-CD20 antibody B-Ly1 (variable region of the murine heavy chain (VH): SEQ ID NO: 1; variable region of the murine light chain (VL): SEQ ID NO: 2—see Poppema, S., and Visser, L., Biotest Bulletin 3 (1987) 131-139) by chimerization with a human constant domain from IgG1 and following humanization (see WO 2005/044859 and WO 2007/031875). These “humanized B-Ly1 antibodies” are disclosed in detail in WO 2005/044859 and WO 2007/031875.

Preferably the “humanized B-Ly1 antibody” has variable region of the heavy chain (VH) selected from group of SEQ ID No. 3 to SEQ ID No. 20 (B-HH2 to B-HH9 and B-HL8 to B-HL17 of WO 2005/044859 and WO 2007/031875). Especially preferred are Seq. ID No. 3, 4, 7, 9, 11, 13 and 15 (B-HH2, BHH-3, B-HH6, B-HH8, B-HL8, B-HL11 and B-HL13 of WO 2005/044859 and WO 2007/031875). Preferably the “humanized B-Ly1 antibody” has variable region of the light chain (VL) of SEQ ID No. 20 (B-KV1 of WO 2005/044859 and WO 2007/031875). Preferably the “humanized B-Ly1 antibody” has a variable region of the heavy chain (VH) of SEQ ID No. 7 (B-HH6 of WO 2005/044859 and WO 2007/031875) and a variable region of the light chain (VL) of SEQ ID No. 20 (B-KV1 of WO 2005/044859 and WO 2007/031875). Furthermore the humanized B-Ly1 antibody is preferably an IgG1 antibody. According to the invention such afocusylated humanized B-Ly1 antibodies are glycoengineered (GE) in the Fc region according to the procedures described in WO 2005/044859, WO 2004/065540, WO2007/031875, Umana, P., et al., Nature Biotechnol. 17 (1999) 176-180 and WO 99/154342. The afucosylated glyco-engineered humanized B-Ly1 (B-HH6-B-KV1 GE) is preferred in one embodiment of the invention. Such glycoengineered humanized B-Ly1 antibodies have an altered pattern of glycosylation in the Fc region, preferably having a reduced level of fucose residues. Preferably the amount of fucose is 60% or less of the total amount of oligosaccharides at Asn297 (in one embodiment the amount of fucose is between 40% and 60%, in another embodiment the amount of fucose is 50% or less, and in still another embodiment the amount of fucose is 30% or less). Furthermore the oligosaccharides of the Fc region are preferably bisected. These glycoengineered humanized B-Ly1 antibodies have an increased ADCC.

The oligosaccharide component can significantly affect properties relevant to the efficacy of a therapeutic glycoprotein, including physical stability, resistance to protease attack, interactions with the immune system, pharmacokinetics, and specific biological activity. Such properties may depend not only on the presence or absence, but also on the specific structures, of oligosaccharides. Some generalizations between oligosaccharide structure and glycoprotein function can be made. For example, certain oligosaccharide structures mediate rapid clearance of the glycoprotein from the bloodstream through interactions with specific carbohydrate binding proteins, while others can be bound by antibodies and trigger undesired immune reactions. (Jenkins, N., et al., Nature Biotechnol. 14 (1996) 975-81).

Mammalian cells are the preferred hosts for production of therapeutic glycoproteins, due to their capability to glycosylate proteins in the most compatible form for human application. (Cumming, D. A., et al., Glycobiology 1 (1991) 115-30; Jenkins, N., et al., Nature Biotechnol. 14 (1996) 975-81). Bacteria very rarely glycosylate proteins, and like other types of common hosts, such as yeasts, filamentous fungi, insect and plant cells, yield glycosylation patterns associated with rapid clearance from the blood stream, undesirable immune interactions, and in some specific cases, reduced biological activity. Among mammalian cells, Chinese hamster ovary (CHO) cells have been most commonly used during the last two decades. In addition to giving suitable glycosylation patterns, these cells allow consistent generation of genetically stable, highly productive clonal cell lines. They can be cultured to high densities in simple bioreactors using serum free media, and permit the development of safe and reproducible bioprocesses. Other commonly used animal cells include baby hamster kidney (BHK) cells, NSO- and SP2/0-mouse myeloma cells. More recently, production from transgenic animals has also been tested. (Jenkins, N., et al., Nature Biotechnol. 14 (1996) 975-981).

All antibodies contain carbohydrate structures at conserved positions in the heavy chain constant regions, with each isotype possessing a distinct array of N-linked carbohydrate structures, which variably affect protein assembly, secretion or functional activity. (Wright, A., and Morrison, S. L., Trends Biotech. 15 (1997) 26-32). The structure of the attached N-linked carbohydrate varies considerably, depending on the degree of processing, and can include high-mannose, multiply-branched as well as biantennary complex oligosaccharides. (Wright, A., and Morrison, S. L., Trends Biotech. 15 (1997) 26-32). Typically, there is heterogeneous processing of the core oligosaccharide structures attached at a particular glycosylation site such that even monoclonal antibodies exist as multiple glycoforms. Likewise, it has been shown that major differences in antibody glycosylation occur between cell lines, and even minor differences are seen for a given cell line grown under different culture conditions. (Lifely, M. R., et al., Glycobiology 5(8) (1995) 813-22).

One way to obtain large increases in potency, while maintaining a simple production process and potentially avoiding significant, undesirable side effects, is to enhance the natural, cell-mediated effector functions of monoclonal antibodies by engineering their oligosaccharide component as described in Umana, P., et al., Nature Biotechnol. 17 (1999) 176-180 and U.S. Pat. No. 6,602,684. IgG1 type antibodies, the most commonly used antibodies in cancer immunotherapy, are glycoproteins that have a conserved N-linked glycosylation site at Asn297 in each CH2 domain. The two complex biantennary oligosaccharides attached to Asn297 are buried between the CH2 domains, forming extensive contacts with the polypeptide backbone, and their presence is essential for the antibody to mediate effector functions such as antibody dependent cellular cytotoxicity (ADCC) (Lifely, M. R., et al., Glycobiology 5 (1995) 813-822; Jefferis, R., et al., Immunol. Rev. 163 (1998) 59-76; Wright, A., and Morrison, S. L., Trends Biotechnol. 15 (1997) 26-32).

It was previously shown that overexpression in Chinese hamster ovary (CHO) cells of β(1,4)-N-acetylglucosaminyltransferase I11 (“GnTII17y), a glycosyltransferase catalyzing the formation of bisected oligosaccharides, significantly increases the in vitro ADCC activity of an antineuroblastoma chimeric monoclonal antibody (chCE7) produced by the engineered CHO cells. (See Umana, P., et al., Nature Biotechnol. 17 (1999) 176-180; and WO 99/154342, the entire contents of which are hereby incorporated by reference). The antibody chCE7 belongs to a large class of unconjugated monoclonal antibodies which have high tumor affinity and specificity, but have too little potency to be clinically useful when produced in standard industrial cell lines lacking the GnTIII enzyme (Umana, P., et al., Nature Biotechnol. 17 (1999) 176-180). That study was the first to show that large increases of ADCC activity could be obtained by engineering the antibody producing cells to express GnTIII, which also led to an increase in the proportion of constant region (Fc)-associated, bisected oligosaccharides, including bisected, non-fucosylated oligosaccharides, above the levels found in naturally-occurring antibodies.

The term “cancer” as used herein refers to cancers or tumors which express the tumor antigen to which the afocusylated antibody is specifically binding. Such cancers includes lymphomas, lymphocytic leukemias, lung cancer, non small cell lung (NSCL) cancer, bronchioloalviolar cell lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, gastric cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, cancer of the bladder, cancer of the kidney or ureter, renal cell carcinoma, carcinoma of the renal pelvis, mesothelioma, hepatocellular cancer, biliary cancer, neoplasms of the central nervous system (CNS), spinal axis tumors, brain stem glioma, glioblastoma multiforme, astrocytomas, schwanomas, ependymonas, medulloblastomas, meningiomas, squamous cell carcinomas, pituitary adenoma, including refractory versions of any of the above cancers, or a combination of one or more of the above cancers.

Preferably the combination treatment of an afocusylated antibody according to the invention in combination with a cytokine selected from human GM-CSF, human M-CSF and/or human IL3 (which all differentiate human monocytes/pericytes into macrophage) is used for the treatment of cancers or tumors which are infiltrated by monocytes/pericytes; and is especially valuable for treatment of cancers or tumors with a high infiltration by monocytes/pericytes. The monocytes/pericytes-infiltration of cancers or tumors can be detected (in the tumor tissue after biopsy) by monocytes/pericyte-specific staining using monocyte-specific markers like CD14 (Wright S. D. et al., Science 249 (1990) 1431-1433; Bogman M. J. et al., Transplant Proc. 23 (1991) 1293-1294; Andreesen, R, et al., J Leukoc Biol. 47(6) (1990) 490-7). Typically, a person skilled in the art will use the combination treatment of an afocusylated antibody according to the invention in combination with a cytokine selected from human GM-CSF, human M-CSF and/or human IL3 for the treatment of monocytes/pericytes-infiltrated cancers or tumors which express the tumor antigen to which the afocusylated antibody is specifically binding. So in one embodiment the cancer is a monocytes/pericytes-infiltrated cancer (detectable by the monocyte specific CD14 antigen).

The term “expression of the CD20” antigen is intended to indicate an significant level of expression of the CD20 antigen in a cell, preferably on the cell surface of a T- or B-Cell, more preferably a B-cell, from a tumor or cancer, respectively, preferably a non-solid tumor. Patients having a “CD20 expressing cancer” can be determined by standard assays known in the art. E.g. CD20 antigen expression is measured using immunohistochemical (IHC) detection, FACS or via PCR-based detection of the corresponding mRNA.

The term “CD20 expressing cancer” as used herein refers to all cancers in which the cancer cells show an expression of the CD20 antigen. Preferably CD20 expressing cancer as used herein refers to lymphomas (preferably B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma) c) marginal zone lymphomas (including extranodal marginal zone B cell lymphoma (Mucosa-associated lymphatic tissue lymphomas, MALT), nodal marginal zone B cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma) f) hairy cell leukemia, g) lymphocytic lymphoma, waldenstrom's macroglobulinemia, h) acute lymphocytic leukemia (ALL), chronic lymphocytic leukemia (CLL)/small lymphocytic lymphoma (SLL), B-cell prolymphocytic leukemia, i) plasma cell neoplasms, plasma cell myeloma, multiple myeloma, plasmacytoma j) Hodgkin's disease.

More preferably the CD20 expressing cancer is a B-Cell Non-Hodgkin's lymphomas (NHL). Especially the CD20 expressing cancer is a Mantle cell lymphoma (MCL), acute lymphocytic leukemia (ALL), chronic lymphocytic leukemia (CLL), B-cell diffuse large cell lymphoma (DLCL), Burkitt's lymphoma, hairy cell leukemia, follicular lymphoma, multiple myeloma, marginal zone lymphoma, post transplant lymphoproliferative disorder (PTLD), HIV associated lymphoma, waldenstrom's macroglobulinemia, or primary CNS lymphoma.

The term “EGFR expressing cancer” as used herein refers to all cancers in which the cancer cells show an expression of the EGFR antigen. The term “IGF-1R expressing cancer” as used herein refers to all cancers in which the cancer cells show an expression of the IGF-1R antigen.

The term “a method of treating” or its equivalent, when applied to, for example, cancer refers to a procedure or course of action that is designed to reduce or eliminate the number of cancer cells in a patient, or to alleviate the symptoms of a cancer. “A method of treating” cancer or another proliferative disorder does not necessarily mean that the cancer cells or other disorder will, in fact, be eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of a patient, is nevertheless deemed to induce an overall beneficial course of action.

The terms “co-administration” or “co-administering” refer to the administration of said afucosylated antibody, preferably the afucosylatedanti-CD20 antibody, anti-EGFR antibody or anti-IGF-1R antibody), and said cytokine selected from human GM-CSF, human M-CSF and/or human IL-3 as one single formulation or as two separate formulations. The co-administration can be simultaneous or sequential in either order, wherein preferably there is a time period while both (or all) active agents simultaneously exert their biological activities. Said afucosylated antibody and said cytokine selected from human GM-CSF, human M-CSF and/or human IL-3 are co-administered either simultaneously or sequentially (e.g. via an intravenous (i.v.) through a continuous infusion (one for the antibody and eventually one for the cytokine selected from human GM-CSF, human M-CSF and/or human IL-3). When both therapeutic agents are co-administered sequentially the dose is administered either on the same day in two separate administrations, or one of the agents is administered on day 1 and the second is co-administered on day 2 to day 7, preferably on day 2 to 4. Thus the term “sequentially” means within 7 days after the dose of the first component (cytokine or antibody), preferably within 4 days after the dose of the first component; and the term “simultaneously” means at the same time. The terms “co-administration” with respect to the maintenance doses of said afucosylated antibody and the cytokine selected from human GM-CSF, human M-CSF and/or human IL-3 mean that the maintenance doses can be either co-administered simultaneously, if the treatment cycle is appropriate for both drugs, e.g. every week. Or the cytokine selected from human GM-CSF, human M-CSF and/or human IL-3 is e.g. administered e.g. every first to third day and said afucosylated antibody is administered every week. Or the maintenance doses are co-administered sequentially, either within one or within several days.

It is self-evident that the antibodies are administered to the patient in a “therapeutically effective amount” (or simply “effective amount”) which is the amount of the respective compound or combination that will elicit the biological or medical response of a tissue, system, animal or human that is being sought by the researcher, veterinarian, medical doctor or other clinician.

The amount of co-administration of said afucosylated antibody and said cytokine selected from human GM-CSF, human M-CSF and/or human IL-3 and the timing of co-administration will depend on the type (species, gender, age, weight, etc.) and condition of the patient being treated and the severity of the disease or condition being treated. Said afucosylated antibody and said cytokine selected from human GM-CSF, human M-CSF and/or human IL-3 are suitably co-administered to the patient at one time or over a series of treatments. Depending on the type and severity of the disease, about 1 μg/kg to 50 mg/kg (e.g. 0.1-20 mg/kg) of said afucosylated antibody and 1 μg/kg to 50 mg/kg (e.g. 0.1-20 mg/kg) of said cytokine selected from human GM-CSF, human M-CSF and/or human IL-3 is an initial candidate dosage for co-administration of both drugs to the patient. If the administration is intravenous the initial infusion time for said afucosylated antibody or said cytokine selected from human GM-CSF, human M-CSF and/or human IL-3 may be longer than subsequent infusion times, for instance approximately 90 minutes for the initial infusion, and approximately 30 minutes for subsequent infusions (if the initial infusion is well tolerated).

The preferred dosage of said afucosylated antibody will be in the range from about 0.05 mg/kg to about 30 mg/kg. Thus, one or more doses of about 0.5 mg/kg, 2.0 mg/kg, 4.0 mg/kg, 10 mg/kg or 30 mg/kg (or any combination thereof) may be co-administered to the patient. The preferred dosage of said cytokine selected from human GM-CSF, human M-CSF and/or human IL-3 will be in the range from 0.01 mg/kg to about 30 mg/kg, e.g. 0.1 mg/kg to 10.0 mg/kg for human GM-CSF, human M-CSF and/or human IL-3. Depending on the on the type (species, gender, age, weight, etc.) and condition of the patient and on the type of afucosylated antibody and cytokine selected from human GM-CSF, human M-CSF and/or human IL-3, the dosage and the administration schedule of said afucosylated antibody can differ from the dosage of cytokine selected from human GM-CSF, human M-CSF and/or human IL-3. E.g. the said afucosylated antibody may be administered e.g. every one to three weeks and said cytokine selected from human GM-CSF, human M-CSF and/or human IL-3 may be administered daily or every 2 to 10 days. An initial higher loading dose, followed by one or more lower doses may also be administered.

In a preferred embodiment, the medicament is useful for preventing or reducing metastasis or further dissemination in such a patient suffering from cancer, preferably from monocytes/pericytes infiltrated cancers. The medicament is useful for increasing the duration of survival of such a patient, increasing the progression free survival of such a patient, increasing the duration of response, resulting in a statistically significant and clinically meaningful improvement of the treated patient as measured by the duration of survival, progression free survival, response rate or duration of response. In a preferred embodiment, the medicament is useful for increasing the response rate in a group of patients.

In the context of this invention, additional other cytotoxic, chemotherapeutic or anti-cancer agents, or compounds that enhance the effects of such agents (e.g. cytokines) may be used in the afucosylated antibody and cytokine ((human GM-CSF, human M-CSF and/or human IL-3) combination treatment of cancer. Such molecules are suitably present in combination in amounts that are effective for the purpose intended. Preferably the said afucosylated antibody and cytokine (human GM-CSF, human M-CSF and/or human IL-3) combination treatment is used without such additional cytotoxic, chemotherapeutic or anti-cancer agents, or compounds that enhance the effects of such agents.

Such agents include, for example: alkylating agents or agents with an alkylating action, such as cyclophosphamide (CTX; e.g. Cytoxan®), chlorambucil (CHL; e.g. Leukeran®), cisplatin (C is P; e.g. Platinol®) busulfan (e.g. Myleran®), melphalan, carmustine (BCNU), streptozotocin, triethylenemelamine (TEM), mitomycin C, and the like; anti-metabolites, such as methotrexate (MTX), etoposide (VP16; e.g. Vepesid®), 6-mercaptopurine (6 MP), 6-thiocguanine (6TG), cytarabine (Ara-C), 5-fluorouracil (5-FU), capecitabine (e.g. Xeloda®), dacarbazine (DTIC), and the like; antibiotics, such as actinomycin D, doxorubicin (DXR; e.g. Adriamycin®), daunorubicin (daunomycin), bleomycin, mithramycin and the like; alkaloids, such as vinca alkaloids such as vincristine (VCR), vinblastine, and the like; and other antitumor agents, such as paclitaxel (e.g. Taxol®) and paclitaxel derivatives, the cytostatic agents, glucocorticoids such as dexamethasone (DEX; e.g. Decadron®) and corticosteroids such as prednisone, nucleoside enzyme inhibitors such as hydroxyurea, amino acid depleting enzymes such as asparaginase, leucovorin and other folic acid derivatives, and similar, diverse antitumor agents. The following agents may also be used as additional agents: arnifostine (e.g. Ethyol®), dactinomycin, mechlorethamine (nitrogen mustard), streptozocin, cyclophosphamide, lomustine (CCNU), doxorubicin lipo (e.g. Doxil®), gemcitabine (e.g. Gemzar®), daunorubicin lipo (e.g. Daunoxome®), procarbazine, mitomycin, docetaxel (e.g. Taxotere®), aldesleukin, carboplatin, oxaliplatin, cladribine, camptothecin, CPT 11 (irinotecan), 10-hydroxy 7-ethyl-camptothecin (SN38), floxuridine, fludarabine, ifosfamide, idarubicin, mesna, interferon beta, interferon alpha, mitoxantrone, topotecan, leuprolide, megestrol, melphalan, mercaptopurine, plicamycin, mitotane, pegaspargase, pentostatin, pipobroman, plicamycin, tamoxifen, teniposide, testolactone, thioguanine, thiotepa, uracil mustard, vinorelbine, chlorambucil. Preferably the afucosylated antibody and cytokine (GM-CSF, M-CSF and IL-3) combination treatment is used without such additional agents.

The use of the cytotoxic and anticancer agents described above as well as antiproliferative target-specific anticancer drugs like protein kinase inhibitors in chemotherapeutic regimens is generally well characterized in the cancer therapy arts, and their use herein falls under the same considerations for monitoring tolerance and effectiveness and for controlling administration routes and dosages, with some adjustments. For example, the actual dosages of the cytotoxic agents may vary depending upon the patient's cultured cell response determined by using histoculture methods. Generally, the dosage will be reduced compared to the amount used in the absence of additional other agents.

Typical dosages of an effective cytotoxic agent can be in the ranges recommended by the manufacturer, and where indicated by in vitro responses or responses in animal models, can be reduced by up to about one order of magnitude concentration or amount. Thus, the actual dosage will depend upon the judgment of the physician, the condition of the patient, and the effectiveness of the therapeutic method based on the in vitro responsiveness of the primary cultured malignant cells or histocultured tissue sample, or the responses observed in the appropriate animal models.

In the context of this invention, an effective amount of ionizing radiation may be carried out and/or a radiopharmaceutical may be used in addition to the afucosylated antibody and cytokine (human GM-CSF, human M-CSF and/or human IL-3) combination treatment of CD20 expressing cancer. The source of radiation can be either external or internal to the patient being treated. When the source is external to the patient, the therapy is known as external beam radiation therapy (EBRT). When the source of radiation is internal to the patient, the treatment is called brachytherapy (BT). Radioactive atoms for use in the context of this invention can be selected from the group including, but not limited to, radium, cesium-137, iridium-192, americium-241, gold-198, cobalt-57, copper-67, technetium-99, iodine-123, iodine-131, and indium-111. Is also possible to label the antibody with such radioactive isotopes. Preferably the afucosylated antibody and cytokine (human GM-CSF, human M-CSF and/or human IL-3) combination treatment is used without such ionizing radiation.

Radiation therapy is a standard treatment for controlling unresectable or inoperable tumors and/or tumor metastases. Improved results have been seen when radiation therapy has been combined with chemotherapy. Radiation therapy is based on the principle that high-dose radiation delivered to a target area will result in the death of reproductive cells in both tumor and normal tissues. The radiation dosage regimen is generally defined in terms of radiation absorbed dose (Gy), time and fractionation, and must be carefully defined by the oncologist. The amount of radiation a patient receives will depend on various considerations, but the two most important are the location of the tumor in relation to other critical structures or organs of the body, and the extent to which the tumor has spread. A typical course of treatment for a patient undergoing radiation therapy will be a treatment schedule over a 1 to 6 week period, with a total dose of between 10 and 80 Gy administered to the patient in a single daily fraction of about 1.8 to 2.0 Gy, 5 days a week. In a preferred embodiment of this invention there is synergy when tumors in human patients are treated with the combination treatment of the invention and radiation. In other words, the inhibition of tumor growth by means of the agents comprising the combination of the invention is enhanced when combined with radiation, optionally with additional chemotherapeutic or anticancer agents. Parameters of adjuvant radiation therapies are, for example, contained in WO 99/60023.

The afucosylated antibodies are administered to a patient according to known methods, by intravenous administration as a bolus or by continuous infusion over a period of time, by intramuscular, intraperitoneal, intracerobrospinal, subcutaneous, intra-articular, intrasynovial, or intrathecal routes. Intravenous or subcutaneous administration of the antibodies is preferred.

The cytokines selected from human GM-CSF, human M-CSF and/or human IL-3 or the combination thereof are administered to a patient according to known methods, e.g. by intravenous administration as a bolus or by continuous infusion over a period of time, by intramuscular, intraperitoneal, intracerobrospinal, subcutaneous, intra-articular, intrasynovial, intrathecal, or peroral routes. Intravenous, subcutaneous or oral administration of the cytokines selected from human GM-CSF, human M-CSF and/or human IL-3 or the combination thereof is preferred.

As used herein, a “pharmaceutically acceptable carrier” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention is contemplated. Supplementary active compounds can also be incorporated into the compositions.

### Pharmaceutical Compositions

Pharmaceutical compositions can be obtained by processing the afucosylated antibodies according to the invention, as e.g. the anti-CD20 antibodies, the anti-EGFR antibodies or the anti-IGF-1R antibodies, and/or the cytokine selected from human GM-CSF, human M-CSF and/or human IL-3 according to this invention with pharmaceutically acceptable, inorganic or organic carriers. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

One embodiment of the invention is composition comprising both said afucosylated antibody with an amount of fucose is 60% or less (preferably said afucosylated anti-CD20 antibody, anti-EGFR antibody or anti-IGF-1R antibody) and one or more cytokines selected from human GM-CSF, human M-CSF and/or human IL-3, for use in the treatment of cancer, in particular of CD20 expressing cancer.

Said pharmaceutical composition may further comprise one or more pharmaceutically acceptable carriers.

The present invention further provides a pharmaceutical composition, in particular for use in cancer, comprising (i) an effective first amount of an afucosylated antibody with an amount of fucose is 60% or less (preferably an afucosylated anti-CD20 antibody, anti-EGFR antibody or anti-IGF-1R antibody)), and (ii) an effective second amount of one or more cytokines selected from human GM-CSF, human M-CSF and/or human IL-3. Such composition optionally comprises pharmaceutically acceptable carriers and/or excipients.

Pharmaceutical compositions of the afucosylated antibody alone used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

Pharmaceutical compositions of the cytokines selected from human GM-CSF, human M-CSF and/or human IL-3, depend on their pharmaceutical properties. Such compositions can be similar to those describe above for the afucosylated antibody.

In one further embodiment of the invention the pharmaceutical compositions according to the invention are preferably two separate formulations for said afucosylated antibody and said cytokine selected from human GM-CSF, human M-CSF and/or human IL-3.

The active ingredients may also be entrapped in microcapsules prepared, for example, by coacervation techniques or by interracial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

The present invention further provides a method for the treatment of cancer, comprising administering to a patient in need of such treatment (i) an effective first amount of an afucosylated antibody with an amount of fucose is 60% or less, (preferably an afucosylated anti-CD20 antibody, anti-EGFR antibody or anti-IGF-1R antibody); and (ii) an effective second amount of one or more cytokines selected from human GM-CSF, human M-CSF and/or human IL-3.

In one embodiment the method is characterized in that the afocusylated antibody shows an increased ADCC.

In one embodiment the method is characterized in that said afucosylated antibody is an anti-CD20 antibody and said cancer is a CD20 expressing cancer.

In one embodiment the method is characterized in that said afucosylated anti-CD20 antibody is a humanized B-Ly1 antibody.

In one embodiment the method is characterized in that said afucosylated antibody is an anti-EGFR antibody and said cancer is an EGFR-expressing cancer.

In one embodiment the method is characterized in that said afucosylated anti-EGFR antibody is a humanized ICR62 antibody.

In one embodiment the method is characterized in that said afucosylated antibody is an anti-IGF-1R antibody and said cancer is an IGF-1R-expressing cancer.

In one embodiment the method is characterized in that said afucosylated anti-IGF-1R antibody is a human HUMAB-Clone 18.

In one embodiment the method is characterized in that said the cancer is a monocytes/pericytes-infiltrated cancer.

In one embodiment the method is characterized in that as cytokine only human GM-CSF is co-administered in said combination treatment.

In one embodiment the method is characterized in that as cytokine only human M-CSF is co-administered in said combination treatment.

In one embodiment the method is characterized in that as cytokine only IL-3 is co-administered in said combination treatment.

In one embodiment the method is characterized in that cytokines only GM-CSF and IL-3 are co-administered in said combination treatment.

In one embodiment the method is characterized in that the cytokines human GM-CSF, human M-CSF and/or human IL-3 are co-administered in said combination treatment.

In one embodiment the method is characterized in that one or more additional other cytotoxic, chemotherapeutic or anti-cancer agents, or compounds or ionizing radiation that enhance the effects of such agents are administered.

As used herein, the term “patient” preferably refers to a human in need of treatment with an afucosylated antibody, preferably an afucosylated anti-CD20 antibody, anti-EGFR antibody or anti-IGF-1R antibody) (e.g. a patient suffering from CD20, EGFR or IGF-1R expressing cancer, respectively) for any purpose, and more preferably a human in need of such a treatment to treat cancer, or a precancerous condition or lesion. However, the term “patient” can also refer to non-human animals, preferably mammals such as dogs, cats, horses, cows, pigs, sheep and non-human primates, among others.

The invention further comprises an afucosylated antibody, preferably an afucosylated anti-CD20 antibody, anti-EGFR antibody or anti-IGF-1R antibody, for the treatment of cancer in combination with one or more cytokines selected from human GM-CSF, human M-CSF and/or human IL-3.

The invention further comprises an afucosylated antibody specifically binding to a tumor antigen (which is CD20, EGFR or IGF-1R, more preferably CD20) with an amount of fucose is 60% or less, and one or more cytokines selected from human GM-CSF, human M-CSF and/or human IL-3 for the treatment of cancer.

In one embodiment that said the cancer is a monocytes/pericytes-infiltrated cancer.

In one embodiment the afocusylated antibody shows an increased ADCC.

In one embodiment said afucosylated antibody is an anti-CD20 antibody and said cancer is a CD20 expressing cancer.

In one embodiment said afucosylated anti-CD20 antibody is a humanized B-Ly1 antibody.

In one embodiment said afucosylated antibody is an anti-EGFR antibody and said cancer is an EGFR-expressing cancer.

In one embodiment said afucosylated anti-EGFR antibody is a humanized ICR62 antibody.

In one embodiment the said afucosylated antibody is an anti-IGF-1R antibody and said cancer is an IGF-1R-expressing cancer.

In one embodiment said afucosylated anti-IGF-1R antibody is a human HUMAB-Clone 18.

In one embodiment the afucosylated antibody (preferably the afucosylated anti-CD20 antibody, anti-EGFR antibody or anti-IGF-1R antibody)) is used in combination with GM-CSF.

In one embodiment the afucosylated antibody (preferably the afucosylated anti-CD20 antibody, anti-EGFR antibody or anti-IGF-1R antibody)) is used in combination with M-CSF.

In one embodiment the afucosylated antibody (preferably the afucosylated anti-CD20 antibody, anti-EGFR antibody or anti-IGF-1R antibody) is used in combination with IL-3.

In one embodiment the afucosylated antibody (preferably the afucosylated anti-CD20 antibody, anti-EGFR antibody or anti-IGF-1R antibody) is used in combination with human GM-CSF, human M-CSF and/or human IL-3.

In one embodiment the afucosylated antibody (preferably the afucosylated anti-CD20 antibody, anti-EGFR antibody or anti-IGF-1R antibody) is used in combination with GM-CSF and IL-3.

Preferably the CD20 expressing cancer is a B-Cell Non-Hodgkin's lymphoma (NHL).

The following examples and figures are provided to aid the understanding of the present invention, the true scope of which is set forth in the appended claims. It is understood that modifications can be made in the procedures set forth without departing from the spirit of the invention.

### SEQUENCE LISTING

SEQ ID NO: 1 amino acid sequence of variable region of the heavy chain (VH) of murine monoclonal anti-CD20 antibody B-Ly1.
SEQ ID NO: 2 amino acid sequence of variable region of the light chain (VL) of murine monoclonal anti-CD20 antibody B-Ly1.
SEQ ID NO: 3-19 amino acid sequences of variable region of the heavy chain (VH) of humanized B-Ly1 antibodies (B-HH2 to B-HH9, B-HL8, and B-HL10 to B-HL17)
SEQ ID NO: 20 amino acid sequences of variable region of the light chain (VL) of humanized B-Ly1 antibody B-KV1
SEQ ID NO: 21-22 amino acid sequences of human GM-CSF and variants
SEQ ID NO: 23-25 amino acid sequences of human GM-CSF and variants
SEQ ID NO: 26-27 amino acid sequences of human GM-CSF and variants
SEQ ID NO: 28 amino acid sequences of variable region of the heavy chain (VH) of human anti-IGF-1R antibodies HUMAB-Clone 18
SEQ ID NO: 29 amino acid sequences of variable region of the light chain (VL) of human anti-IGF-1R antibodies HUMAB-Clone 18
SEQ ID NO: 30 amino acid sequences of variable region of the heavy chain (VH) of humanized anti-EGFR ICR62
SEQ ID NO: 31 amino acid sequences of variable region of the light chain (VL) of humanized anti-EGFR ICR62

### BRIEF DESCRIPTION OF THE FIGURES

FIGS. 1a and 1b FACS analysis of co-cultured pericytes/monocytes cells with tumor cells—

FIG. 1a in the presence of 10 μg/ml afucosylated glycoengineered humanized B-Ly1 (B-HH6-B-KV1 GE)

FIG. 1b in the presence of 10 μg/ml human IgG (Isotype),

FIG. 2 Peritoneal effector cell response (pericytes/monocytes were stimulated with mGM-CSF/mG-GCS/mL-3) on SU-DHL4 tumor cells:
A=10 μg/ml Rituximab
B=10 μg/ml afucosylated glycoengineered humanized B-Ly1 (B-HH6-B-KV1 GE)
C=untreated
D=Human IgG,

FIG. 3 Study of tumor cell elimination (10 μg/ml Rituximab were used),

FIG. 4 Antitumor efficacy of Effector:target ratio titration (afocusylated humanized B-Ly1 antibody B-HH6-B-KV1 GE (=Squares) and non-afocusylated Rituximab (=triangles) were used at 10 μg/ml),

FIGS. 5a and 5b Comparison of antitumor efficacy/elimination of tumor cells (SU-DHL4 (Diffuse Large Cell Lymphoma cell) of afocusylated glycoengineered antibodies (B-HH6-B-KV1 GE) compared to non-afocusylated antibodies (wt B-HH6-B-KV1 and Rituximab) at different antibody concentrations at an Effector:target ratio (E:T) of 1:1 (FIG. 5a) and 3:1 (FIG. 5b)). From both figures the higher efficacy of afocusylated glycoengineered antibody (B-HH6-B-KV1 GE) becomes clear—especially when compared to the wild type (wt) parent antibody (wt B-HH6-B-KV1),

FIG. 6 Antitumor efficacy/Elimination of tumor cells (SU-DHL4 (Diffuse Large Cell Lymphoma cell) and Z-138 (Mantle Cell Lymphoma)) by human macrophages during the treatment with human M-CSF and a) afocusylated humanized B-Ly1 antibody B-HH6-B-KV1 GE b) non-afocusylated (non-glycoengineered) Rituximab at 10 μg/ml; or c) without antibody (as well as controls),

FIG. 7 Comparison of antitumor efficacy/elimination on A431 tumor cells by human macrophages during the treatment with human M-CSF and
afocusylated anti-EGFR humanized ICR62 at 10 μg/ml, or
wt non-afocusylated (non-glycoengineered) anti-EGFR humanized ICR62 at 10 μg/ml;

FIG. 8 Comparison of antitumor efficacy/elimination on H322M tumor cells during the treatment with human M-CSF and
afocusylated anti-IGF-1R HUMAB-Clone 18 at 10 μg/ml; or
wt non-afocusylated (non-glycoengineered) anti-IGF-1R HUMAB-Clone 18 at 10 μg/ml;

### EXPERIMENTAL PROCEDURES

### Material and Methods

1. Cell culture

The human Diffuse Large Cell Lymphoma cell lines OCI-Ly19, SU-DHL4, Namalwa and the human Mantle Cell Lymphoma line Z-138 were maintained in culture in RPMI-1640 medium (PanBiotech GmbH, Cat.-No. PO₄-18500) containing 10% FCS (Gibco, Cat.-No.10500-064)) and 2 mM L-glutamine (PAA, Cat.-No. M11-004).

The human epidermoid carcinoma cell line A431 and the human NSCLC cell line H322M were maintained in RPMI-1640 medium (PanBiotech GmbH, Cat.-No. PO4-18500) containing 10% FCS (Gibco, Cat.-No. 10500-064) and 2 mM L-glutamine (PAA, Cat.-No. M11-004).

### 2. Antibodies and Reagents

Antibodies used are listed in Table 1 (see below).

TABLE 1
Specification of antibody or antibody fragments

| | | | Amount of |
|---|---|---|---|
| Name | Concentration | | fucose |
| afocusylated, humanized B-Ly1 antibody | 10 | mg/ml | ~45% |
| B-HH6-B-KV1 GE (glycoengineered) | | | |
| Rituximab (Rx) (non-afocusylated; | 10 | mg/ml | >85% |
| nonglycoengineered) | | | |
| non-afocusylated humanized B-Ly1 | 12.5 | mg/ml | >85% |
| antibody B-HH6-B-KV1 (non- | | | |
| glycoengineered) | | | |
| F(ab)2 of humanized B-Ly1 antibody B- | 5 | mg/ml | — |
| HH6-B-KV1 GE | | | |
| F(ab)2 of Rituximab | 5 | mg/ml | — |
| Fab of humanized B-Ly1 antibody B- | 0.4 | mg/ml | — |
| HH6-B-KV1 GE | | | |
| wild type anti-EGFR humanized ICR62 | 7.7 | mg/ml | >85% |
| (non-afocusylated) | | | |
| afocusylated anti-EGFR humanized | 10.3 | mg/ml | ~35% |
| ICR62 | | | |
| wild type anti-IGF-1R HUMAB-Clone 18 | 25.6 | mg/ml | >85% |
| (non-afocusylated) | | | |
| afocusylated anti-IGF-1R HUMAB-Clone | 15.0 | mg/ml | ~35% |
| 18 | | | |
| hu IgG | 11 | mg/ml | >85% |

### 3. Preparation of Peritoneal Monocytes

Briefly, SCID beige mice were killed by cervical dislocation and 5 ml ice cold PBS was injected into the peritoneum. After massaging for 1-2 minutes, recover as much as possible (max. 4 ml) of peritoneal lavage out of the peritoneal cavity—results in a yield of—10⁶ peritoneal cells per mouse. After centrifugation, resuspend the cells with 1 ml complete culture medium (provider as above: RPMI-1640+10% FCS+2 mM Glutamine) and seed 100 μl/well into a 96 well round bottom plate. Stimulation of the pericytes/monocytes was performed with cytokines (table 2) for 3 days (till 7 days possible) before using them for effector functional assays.

### 4. Used Cytokines and Concentrations

TABLE 2
Cytokines and respective concentrations used for
pericyte/monocyte stimulation in all experiments

| Cytokine | Concentration | Supplier |
|---|---|---|
| mGM-CSF | 10 ng/ml | Sigma Order No. G0282 |
| mIL-3 | 10 ng/ml | Sigma Order No. I4144 |
| mIL-4 | 10 ng/ml | Sigma Order No. I1020 |
| mM-CSF | 20 ng/ml | Sigma Order No. M9170 |
| hGM-CSF | 10 ng/ml | Sigma Order No. G5035 |
| hIL-3 | 10 ng/ml | Sigma Order No. I1646 |
| hM-CSF | 20 ng/ml | Sigma Order No. M6518 |

5. Co-Culture of Pericytes/Monocytes with Tumor Cells

2×10⁴ isolated pericytes/monocytes were seeded per well (100 μl) into a 96well round-bottom plate and cultured with the respective cytokine for 3-7 days. Then 5×10³ tumor cells and the respective antibody were added (100 μl) and incubated for up to 3 days (depending on the experiment) until analysis.

6. Preparation of Human Monocytes and Co-Culture with Tumor Cells

Briefly, 100 ml EDTA-blood (EDTA reduced to 1 mM) was used to isolate monocytes. The “RosetteSep Kit” from StemCell Tech. Inc. (Cat.15068) was applied and monocytes were stimulated with 20 ng/ml hM-CSF in RPMI-1640 medium (PanBiotech GmbH, Cat.-No. PO4-18500)+10% FCS (Gibco, Cat.-No. 10500-064)+4 mM Glutamin (Sigma Order No. G7513)+50 ng/ml IGF-R3-long (JRH Biosciences, Pdt Code 85580). Cells were washed and diluted to ˜2×10⁵ cells/ml. 100 μl were seeded into a 96-well round bottom plate with the a.m. culture medium for 2 days, then 100 μl culture medium was added for another 4 days. Tumor cells and the respective antibody were added after removal of 100 μl supernatant (50 μl of a SU-DHL4 suspension with 10⁵ cells/ml). 3 days later FACS analysis of the co-culture was performed.

### 7. FACS Analysis

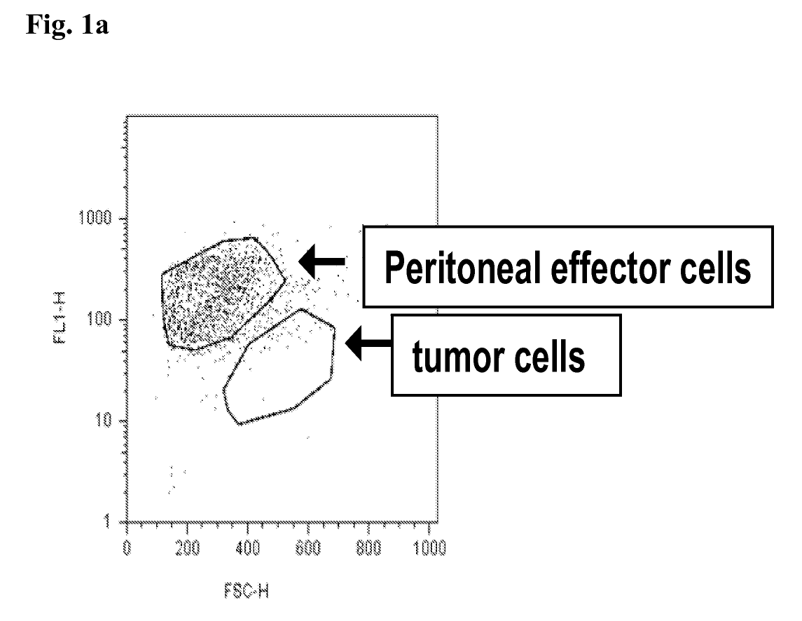

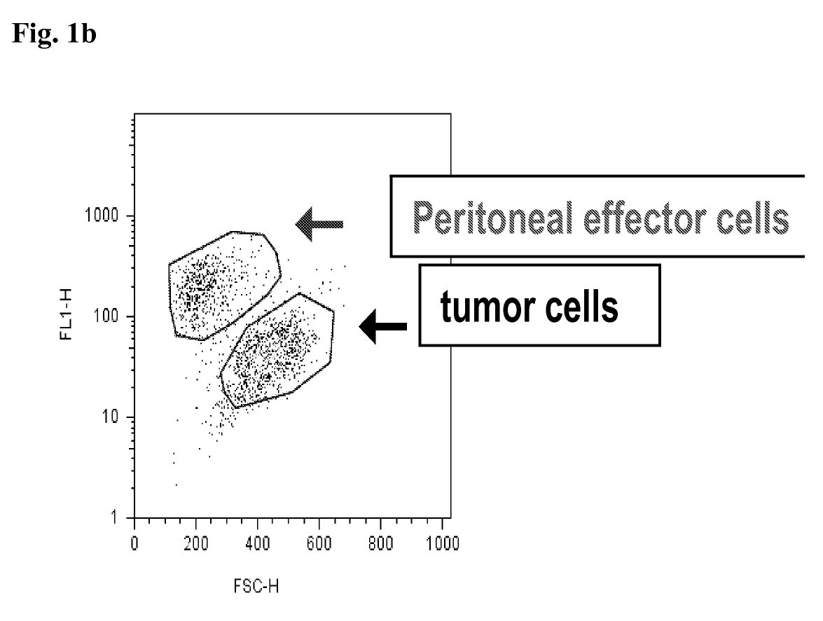

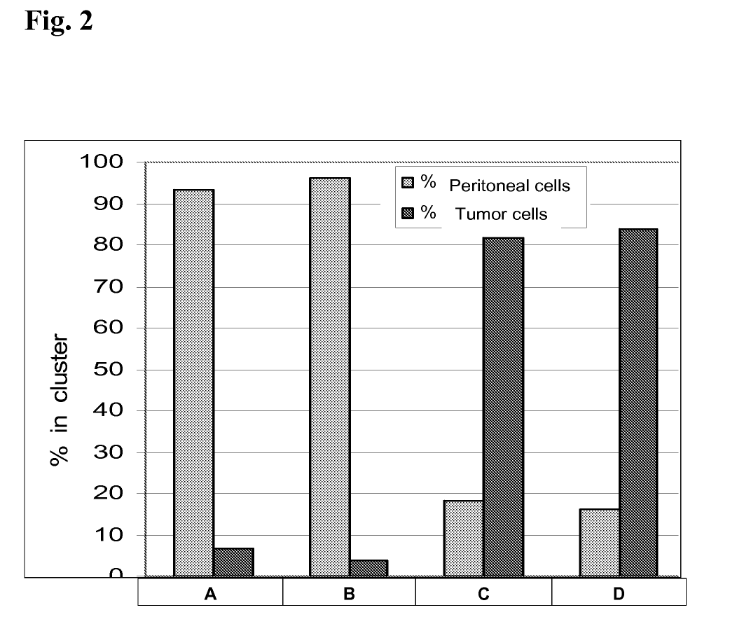

The co-cultured cells were resuspended in the wells and then transferred into tubes, centrifuged at 400×g and then resuspended in 300 μl culture medium containing 10 μg/ml PI. The tubes were kept on ice until analysis. From the FSC versus FL-3H (PI) dotplot, the viable cells were gated and analyzed. Within the viable population, the FCS versus FL-1H auto fluorescence was analyzed for calculating the percentage of tumor and effector cells. FIG. 1 shows dotplots for the differential analytics by FACS.

### Example 1

### Stimulation of Peritoneal Monocytes with Different Cytokines and their Specific Effect on Tumor Cell Killing

In the first experiments, a combination of cytokines was used, which granted growth of peritoneal cells. FIG. 2 shows, that the pericytes/monocytes stimulated with mGM-CSF/mG-GCS/mIL-3, which support differentiation of murine monocytes and growth of granulocytes and macrophages, were able to eliminate tumor cells effectively.

### FIG. 2: Peritoneal Effector Cell Response on SU-DHL4 Cells

In most of the experiments, the above mentioned mGM-CSF/mG-GCS/mL-3 stimulation was chosen, although in later experiments, also single cytokine stimulation resulted in the same efficacy.

Table 3 summarizes the cytokines tested as single stimulator or in combinations. Effects are characterized as ++, when ≧90%, +, when ≧20% and −, when no tumor cells were eliminated. In all experiments 10 μg/ml anti-CD20 antibodies (afucosylated glycoengineered humanized B-Ly1 (B-HH6-B-KV1 GE), non-afucosylated, non-glycoengineered humanized B-Ly1 (B-HH6-B-KV1) or rituximab) were used.

TABLE 3
Cytokines used for the stimulation of murine
pericytes in order to kill SU-DHL4 cells

| | with afocusylated | without |
|---|---|---|
| | anti-CD20 | afocusylated anti- |
| Cytokine | antibody | CD20 antibody |
| mGM-CSF + mM-CSF + mIL-3 | ++ | − |
| mGM-CSF + mIL-3 | ++ | − |
| mGM-CSF + mIL-4 | ++ | ++ |
| mM-CSF | ++ | − |
| mGM-CSF | ++ | − |
| mIL-3 | ++ | − |
| isotype | − | − |
| without antibody | − | − |

As table 3 figures out, the murine pericytes could be stimulated by mIL-3, mM-CSF and mGM-CSF alone or in combination to finally eliminate tumor cells, when combined with afocusylated, humanized B-Ly1 antibody B-HH6-B-KV1 GE or Rituximab. mIL-4 stimulation mediated antibody independent killing of the tumor cells. Without antibody or using isotype antibody, these cytokines did not result in tumor cell elimination. Analougously the human cytokines GM-CSF, M-CSF, and IL-3 have an high effect on tumor cell killing when combined with an afocusylated antibody according to th invention (see e.g. Example 7). The combination mGM-CSF and mIL-3 was used in most of the following experiments described here.

### Example 2

### CD20 Specific Tumor Cell Killing

CD20 positive cell lines (SU-DHL4 and Z-138) as well as CD20 negative cell lines (OCI-Ly19 and Namalwa) were used to show, that the antibody mediated killing of tumor cells by stimulated pericytes/monocytes is target specific. Table 4 summarizes the results of the experiment, in which the pericytes were stimulated by mGM-CSF+mIL3.

TABLE 4
Killing of CD20-positive tumor cells
by stimulated pericytes/monocytes

| | | | effector | |
|---|---|---|---|---|
| | | | response | |
| | | CD20 | B-HH6-B-KV1 | CD20 |
| | histological | positive/ | GE/ | molecules/ |
| cell line | Type | negative | Rituximab | cell |
| SU-DHL4 | Diffuse Large | positive | +/+ | 1.018.427 |
| | Cell Lymphoma | | | |
| Z138 | Mantle Cell | positive | +/+ | 63.645 |
| | Lymphoma | | | |
| Oci-Ly19 | Diffuse Large | negative | −/n.d. | − |
| | Cell Lymphoma | | | |
| | Burkitt | | | |
| Namalwa | Lymphoma | negative | −/n.d. | − |

Table 4 illustrates, that only CD20 positive cells showed antibody mediated killing.

### Example 3

### Contribution of Fc Part of the Antibody

SU-DHL4 cells were co-cultured with pericytes/monocytes stimulated by mGM-CSF+mIL3 and 10 μg/ml of the respective antibody or fragment were added during co-cultivation. Effects are characterized as ++, when ≧90%, +, when ≧20% and +/−, when there was a minor effect but not zero and −, when no tumor cells were eliminated. The effects are summarized in table 5.

TABLE 5
mediation of killing effects by fragments or whole antibodies

| Antibody | Response |
|---|---|
| Rituximab | ++ |
| afucosylated glycoengineered humanized B-Ly1 (B- | ++ |
| HH6-B-KV1 GE) | |
| non-afucosylated, non-glycoengineered humanized | ++ |
| B-Ly1 (B-HH6-B-KV1) | |
| F(ab)2 humanized B-Ly1 (B-HH6-B-KV1 GE) | +/− |
| Fab humanized B-Ly1 (B-HH6-B-KV1 GE) | − |
| irrelevant IgG | − |
| no antibody | − |

SU-DHL4 cells were effectively eliminated by stimulated pericytes/monocytes, when whole antibodies were used. F(ab)₂ fragments worked to a minor degree, which might reflect the apoptosis/anti-proliferative contribution inherent in the binding characteristics of the antibody. Fab fragments and irrelevant antibodies did not affect tumor cells at all, showing that the antibody Fc-part is required for monocyte mediated cell killing.

### Example 4

### Kinetic Study

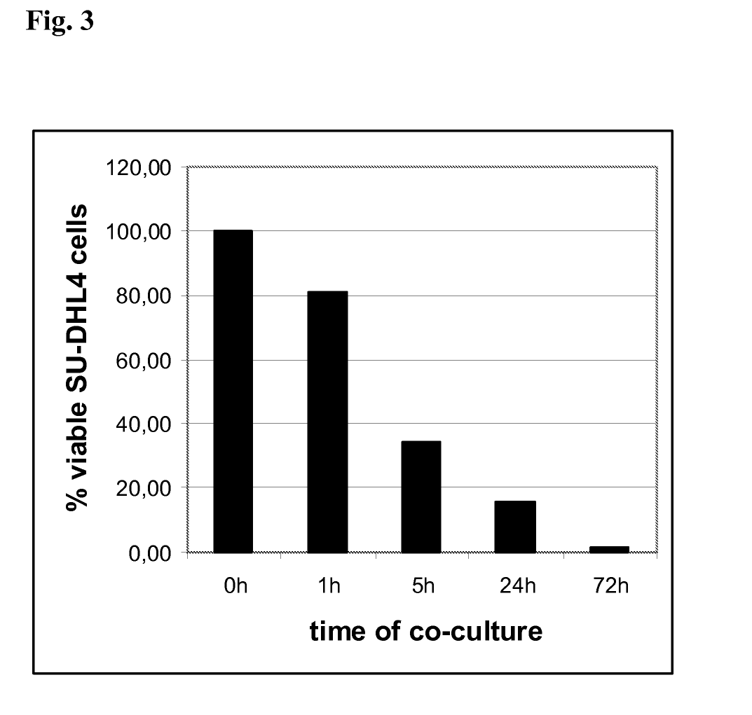

SU-DHL4 cells were co-cultured with pericytes stimulated by mGM-CSF+mIL3 for different time intervals with 10 μg/ml. Only the non-glycoengineered Rituximab was applied for these experiments, however, comparable data are expected for afucosylated glycoengineered humanized B-Ly1 (B-HH6-B-KV1 GE). FIG. 3 shows the percentage of remaining viable tumor cells at different time points.

FIG. 3: Kinetic Study of Tumor Cell Elimination (10 μg/ml Rituximab was Used).

FIG. 3 shows that the elimination of tumor cells is >50% after 5 h and is almost completed over a period of 72 h treatment.

### Example 5

### Elaboration of the Effector:Target (E:T) Ratio Needed for Efficacy

SU-DHL4 cells were co-cultured with pericytes/monocytes stimulated by mGM-CSF+mIL3 in different ratios. In this experiment 10 μg/ml afucosylated glycoengineered humanized B-Ly1 (B-HH6-B-KV1 GE) and Rituximab were used.

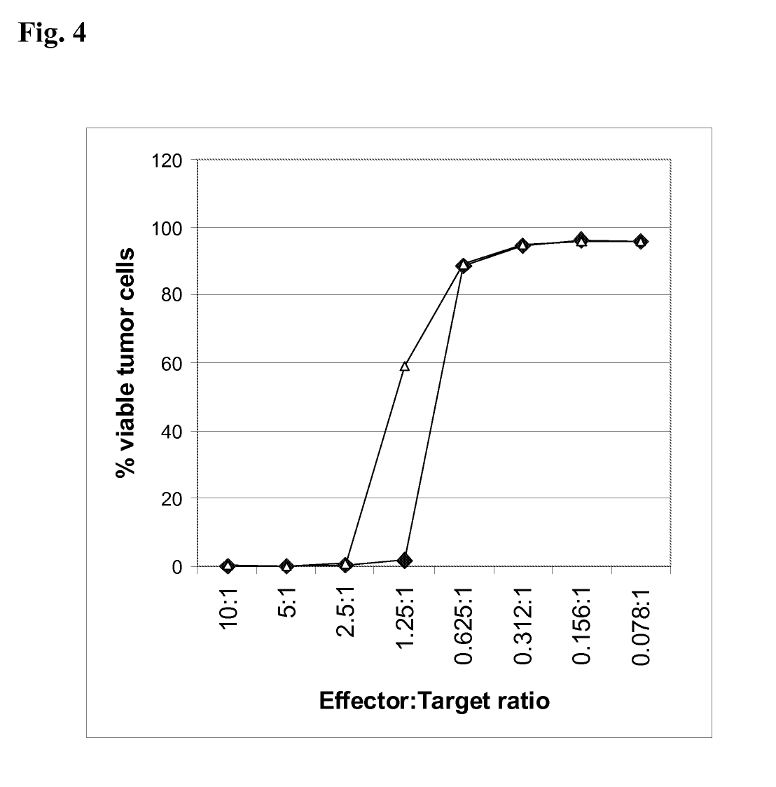

FIG. 4: Effector/Target Ratio (E:T) Titration (Afucosylated Glycoengineered Humanized B-Ly1 (B-HH6-B-KV1 GE) and Rituximab were Used at 10 μg/ml).

As shown in FIG. 4, afucosylated glycoengineered humanized B-Ly1 (B-HH6-B-KV1 GE) totally eliminated tumor cells at an E:T ratio of 1.25:1, whereas Rituximab was able to eliminate 40% tumor cells at this ratio only. This shows that afucosylated glycoengineered humanized B-Ly1 (B-HH6-B-KV1 GE) is superior to Rituximab. Titration of the antibody at a fixed E:T ratio should support these results.

### Example 6

### Tumor Cell Elimination—Treatment of Different Antibodies in Combination with mGM-CSF+mIL3 During Co-Culture of Tumor Cells with Pericytes/Monocytes—Antibody Titration at a Fixed Effector:Target Ratio (E:T Ratio)

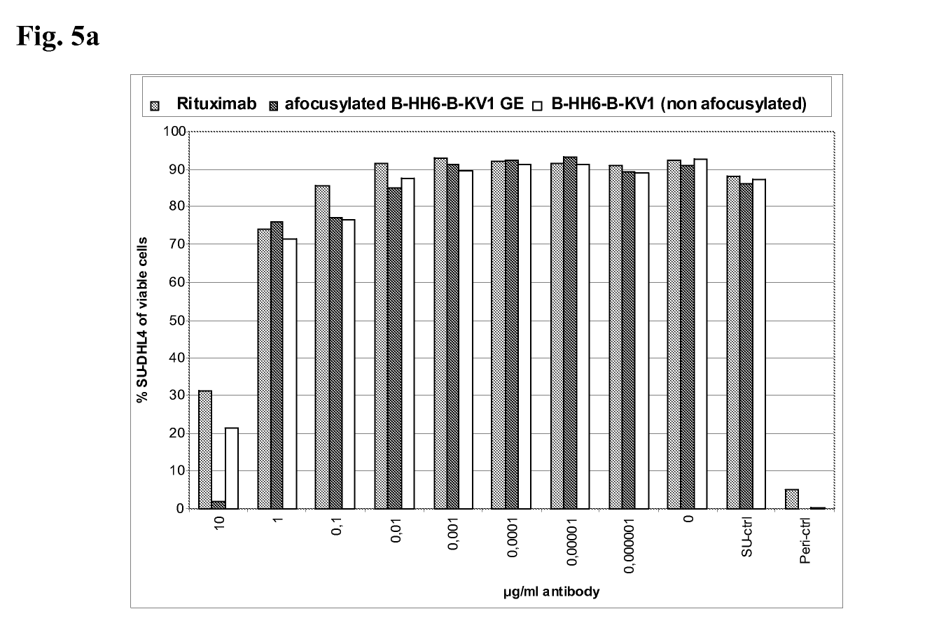

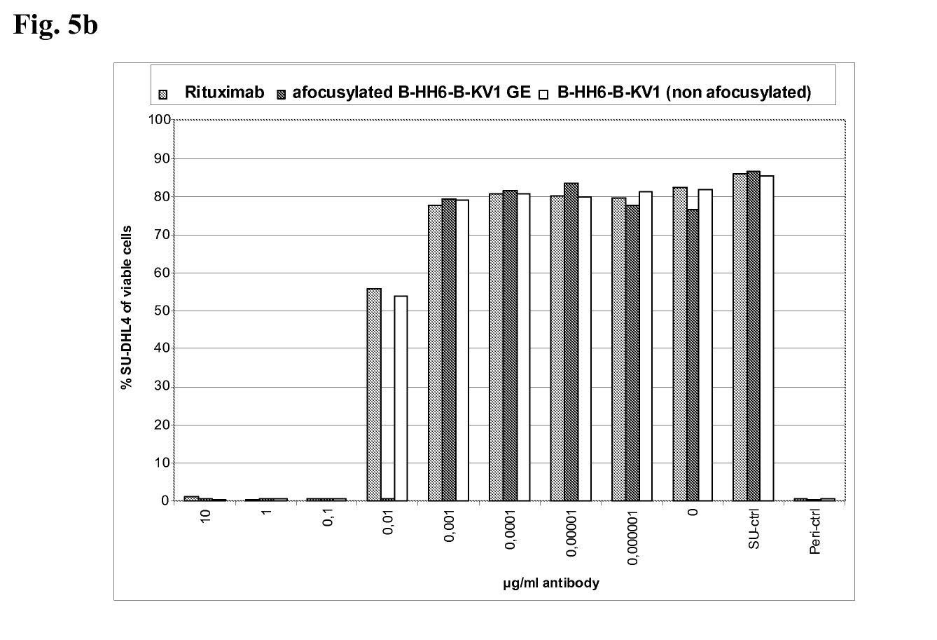

SU-DHL4 cells were co-cultured with pericytes/monocytes stimulated by mGM-CSF+mIL3 in fixed ratios and antibodies were titrated. FIG. 5 shows the percentage of viable tumor cells at E:T ratios 3:1 and 1:1.

### FIG. 5: Efficacy of Different Antibody Concentrations at E:T Ratio a)1:1 and B) 3:1

As shown in FIG. 5, already lower concentrations of afucosylated glycoengineered humanized B-Ly1 (B-HH6-B-KV1 GE) antibody completely eliminate SU-DHL4 cells. Additionally, the concentration of the antibody could be reduced, when higher numbers of effector cells are present. Both non-afucosylated antibodies (non-glycoengineered humanized B-Ly1 with an amount of fucose >80% (B-HH6-B-KV1) and Rituximab) show less efficacy (both have comparable efficacies). Since afucosylated glycoengineered humanized B-Ly1 (B-HH6-B-KV1 GE) and non-afucosylated, non-glycoengineered humanized B-Ly1 (B-HH6-B-KV1) only differ in their degree of fucosylation and consequently in their affinity to the FcγIIIA receptor, the superior efficacy of afucosylated glycoengineered humanized B-Ly1 (B-HH6-B-KV1 GE) over non-afucosylated, non-glycoengineered humanized B-Ly1 (B-HH6-B-KV1) is based on the better recruitment of effector cells. This strongly supports that afocusylated antibodies specifically binding to the CD20 tumor antigen mediate enhanced effector cell mediated killing by peritoneal monocytes.

### Example 7

### Tumor Cell Elimination—Treatment of Different Antibodies in Combination with hM-CSF During Co-Culture of Tumor Cells with Human Pericytes/Monocytes—Efficacy of Human Macrophages as Effectors

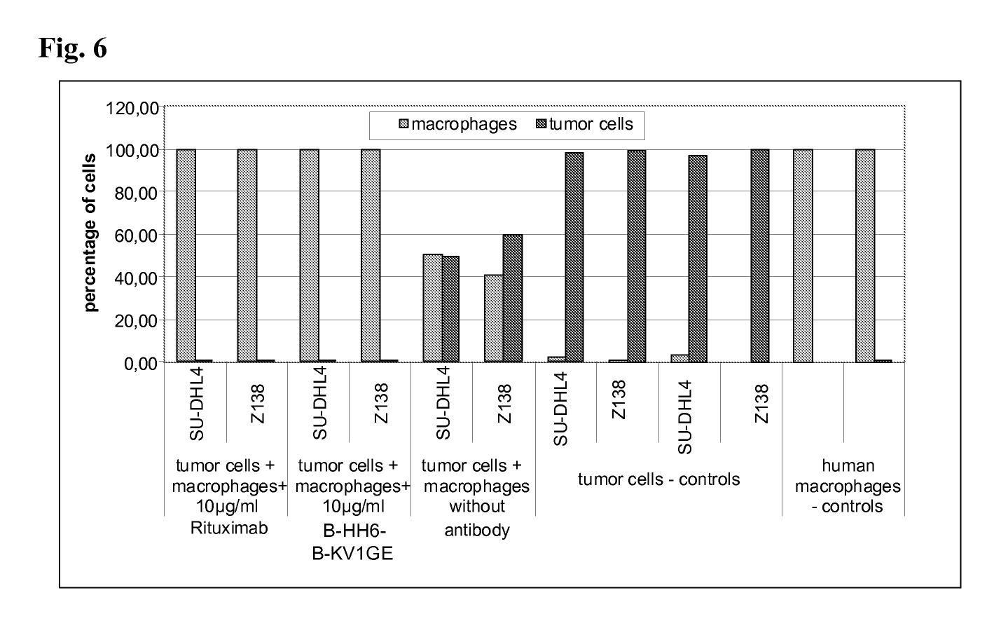

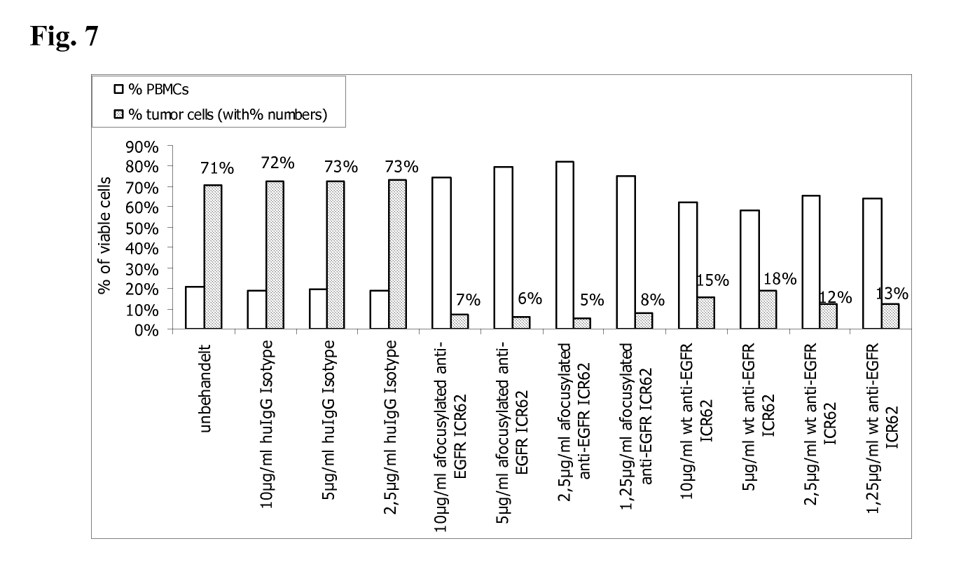

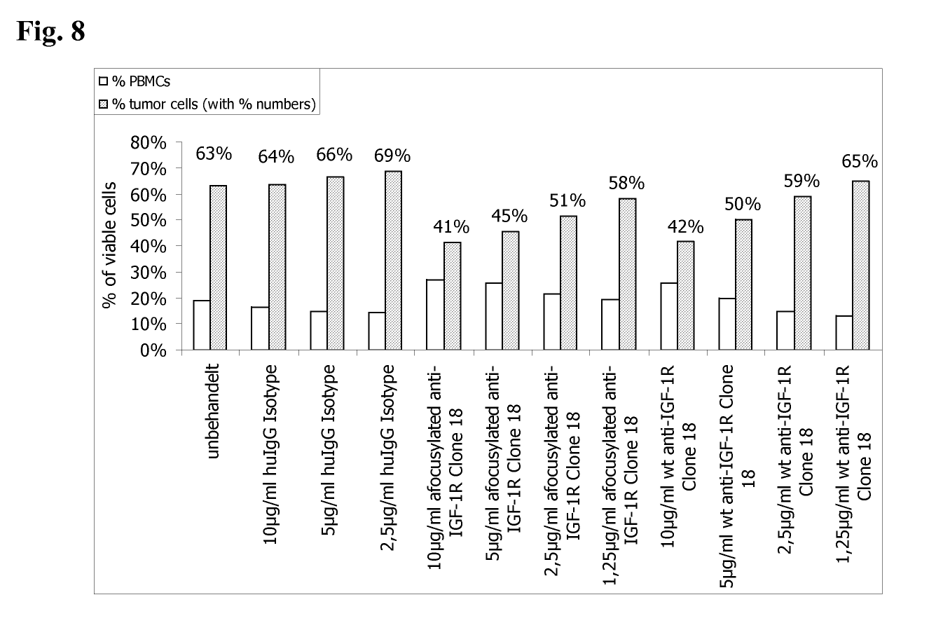

In order to translate these findings into the human setting, human monocytes were isolated out of blood and stimulated by hM-CSF to induce differentiation into macrophages. SU-DHL4 cells were then co-cultured with 10 μg/ml afucosylated glycoengineered humanized B-Ly1 (B-HH6-B-KV1 GE) or Rituximab for 3 days and elimination of the tumor cells was analyzed as shown in FIG. 6.

### FIG. 6: Elimination of Tumor Cells by Human Macrophages

As depicted in FIG. 6, human macrophages are also able to eliminate tumor cells (SU-DHL4 and Z138) upon antibody treatment. At the chosen concentration of 10 μg/ml, there is a saturation of cellular binding of Rituximab and afucosylated glycoengineered humanized B-Ly1 (B-HH6-B-KV1 GE), so that no differentiation between the antibodies could be expected.

### Example 8

### Analysis of Glycostructure of Antibody

For determination of the relative ratios of fucose- and non-fucose (a-fucose) containing oligosaccharide structures, released glycans of purified antibody material are analyzed by MALDI-Tof-mass spectrometry. For this, the antibody sample (about 50 μg) is incubated over night at 37° C. with 5mU N-Glycosidase F (Prozyme# GKE-5010B) in 0.1M sodium phosphate buffer, pH 6.0, in order to release the oligosaccharide from the protein backbone. Subsequently, the glycan structures released are isolated and desalted using NuTip-Carbon pipet tips (obtained from Glygen: NuTip1-10 μl, Cat.Nr#NT1CAR). As a first step, the NuTip-Carbon pipet tips are prepared for binding of the oligosaccharides by washing them with 3 μL 1M NaOH followed by 20 μL pure water (e.g. HPLC-gradient grade from Baker, # 4218), 3 μL 30% v/v acetic acid and again 20 μl pure water. For this, the respective solutions are loaded onto the top of the chromatography material in the NuTip-Carbon pipet tip and pressed through it. Afterwards, the glycan structures corresponding to 10 μg antibody are bound to the material in the NuTip-Carbon pipet tips by pulling up and down the N-Glycosidase F digest described above four to five times. The glycans bound to the material in the NuTip-Carbon pipet tip are washed with 20 μL pure water in the way as described above and are eluted stepwise with 0.5 μL 10% and 2.0 μL 20% acetonitrile, respectively. For this step, the elution solutions are filled in a 0.5 mL reaction vails and are pulled up and down four to five times each. For the analysis by MALDI-T of mass spectrometry, both eluates are combined. For this measurement, 0.4 μL of the combined eluates are mixed on the MALDI target with 1.6 μL SDHB matrix solution (2,5-Dihydroxybenzoic acid/2-Hydroxy-5-Methoxybenzoic acid [Bruker Daltonics #209813] dissolved in 20% ethanol/5 mM NaCl at 5 mg/ml) and analysed with a suitably tuned Bruker Ultraflex TOF/TOF instrument. Routinely, 50-300 shots are recorded and summed up to a single experiment. The spectra obtained are evaluated by the flex analysis software (Bruker Daltonics) and masses are determined for the each of the peaks detected. Subsequently, the peaks are assigned to fucose or a-fucose (non-fucose) containing glycol structures by comparing the masses calculated and the masses theoretically expected for the respective structures (e.g. complex, hybride and oligo- or high-mannose, respectively, with and without fucose).

For determination of the ratio of hybride structures, the antibody sample are digested with N-Glycosidase F and Endo-Glycosidase H concomitantly N-glycosidase F releases all N-linked glycan structures (complex, hybride and oligo- and high mannose structures) from the protein backbone and the Endo-Glycosidase H cleaves all the hybride type glycans additionally between the two GlcNAc-residue at the reducing end of the glycan. This digest is subsequently treated and analysed by MALDI-T of mass spectrometry in the same way as described above for the N-Glycosidase F digested sample. By comparing the pattern from the N-Glycosidase F digest and the combined N-glycosidase F/Endo H digest, the degree of reduction of the signals of a specific glyco structure is used to estimate the relative content of hybride structures.

The relative amount of each glyco structure is calculated from the ratio of the peak height of an individual glycol structure and the sum of the peak heights of all glyco structures detected. The amount of fucose is the percentage of fucose-containing structures related to all glyco structures identified in the N-Glycosidase F treated sample (e.g. complex, hybride and oligo- and high-mannose structures, resp.). The amount of afucosylation is the percentage of fucose-lacking structures related to all glyco structures identified in the N-Glycosidase F treated sample (e.g. complex, hybride and oligo- and high-mannose structures, resp.).

### Example 9

### Tumor Cell Elimination—Treatment of Different Afocusylated Antibodies (Against CD20, EGFR and IGF-R) in Combination with hM-CSF During Co-Culture of Different Tumor Cells with Human Pericytes/Monocytes

In order to show the that combination of pericyte/monocyte-stimulating cytokines e.g. hM-CSF with different afocusylated antibodies leads to enhanced tumor cell death in tumor cell-human preicytes/monocytes cocultures (which reflect human in vivo settings) compared to the combination of non-glycoengineered (non-afocyslated) parent antibodies with these cytokines, human monocytes were isolated out of blood and stimulated by hM-CSF to induce differentiation into macrophages.

A) A431 cells and stimulated monocytes were then co-cultured with declining concentrations of antibody starting with 10 μg/ml afucosylated glycoengineered <EGFR> GA201 or the non-glycoengineered (non-afocyslated) parent antibody for 3 days and elimination of the tumor cells was analyzed as shown in FIG. 7.

B) H322M cells and stimulated monocytes were then co-cultured with declining concentrations of antibody starting with 10 μg/ml afucosylated glycoengineered <IGF-1R> HUMAB-Clone 18 or the non-glycoengineered (non-afocyslated) wild type parent antibody <IGF-1R> HUMAB-Clone 18 for 3 days and elimination of the tumor cells was analyzed as shown in FIG. 8.

What is claimed is:

1. A method for the treatment of cancer, comprising administering to a patient in need of such treatment (i) an afucosylated antibody with an amount of fucose is 60% or less; and (ii) one or more cytokines selected from the group consisting of human GM-CSF, human M-CSF and human IL-3.

2. The method of claim 1 wherein said afucosylated antibody is an anti-CD20 antibody and said cancer is a CD20-expressing cancer.

3. The method according to claim 2, wherein said afucosylated anti-CD20 antibody is a humanized B-Ly1 antibody.

4. The method of claim 1 wherein said afucosylated antibody is an anti-EGFR antibody and said cancer is an EGFR-expressing cancer.

5. The method of claim 4, wherein said afucosylated anti-EGFR antibody is a humanized ICR62 antibody.

6. The method of claim 1 wherein said afucosylated antibody is an anti-IGF-1R antibody and said cancer is an IGF-1R-expressing cancer.

7. The method of claim 6, wherein said afucosylated anti-IGF-1R antibody is a human HUMAB-Clone 18.

8. The method of claim 1 wherein said the cancer is a monocytes/pericytes-infiltrated cancer.

9. The method of claim 1 wherein said afocusylated antibody shows an increased ADCC.

10. The method of claim 1 wherein said one or more cytokines is only human GM-CSF.

11. The method of claim 1 wherein said one or more cytokines is only human M-CSF.

12. The method of claim 1 wherein said one or more cytokines is only IL-3.

13. The method of claim 1 wherein said one or more cytokines is GM-CSF and IL-3.

14. The method of claim 1 wherein said one or more cytokines is human GM-CSF, human M-CSF and human IL-3.

15. The method of claim 1 wherein one or more additional other cytotoxic, chemotherapeutic, or anti-cancer agents, or compounds or ionizing radiation that enhance the effects of such agents are administered.

16. A composition comprising an afucosylated antibody specifically binding to a tumor antigen with an amount of fucose of 60% or less and one or more cytokines selected from the group consisting of human GM-CSF, human M-CSF and human IL-3.
